(19) United States
(12) Patent Application Publication
(10) Pub. No.: US 2005/0232896 A1
(43) Pub. Date: Oct. 20, 2005

(54) Cd137 as a proliferation factor for hematopoietic stem cells

(76) Inventor: Herbert Schwarz, Singapore (SG)

Correspondence Address: BROWDY AND NEIMARK, P.L.L.C.;624 NINTH STREET, NW, SUITE 300, WASHINGTON, DC, 20001-5303, US

(21) Appl. No.: 10/491,586

(22) PCT Filed: Oct. 3, 2002
(86) PCT No.: PCT/EP02/11101

§ 371 Date: Apr. 2, 2004

(30) Foreign Application Priority Data
Oct. 4, 2001 (EP) 01123709.6

(51) Int. Cl. A61K 38/19; A61K 38/17; C07K 14/74

(52) U.S. Cl. 424/85.1; 514/12; 530/350

(57) ABSTRACT

The invention provides the CD137 molecule or a functional analogue thereof, for use in stimulating hematopoiesis. The invention further provides a method of treatment of a disorder characterized by insufficient numbers of cells of the hematopoietic system, including but not limited to T cells, B cells, granulocytes, macrophages, mesenchymal cells, osteoclasts and multipotent adult progenitor cells, comprising the application of CD137 or a functional analogue thereof.

### FIELD OF THE INVENTION

The invention relates to the field of hematopoietic growth factors; in particular, the invention relates to the induction of growth of hematopoietic stem cells.

### BACKGROUND OF THE INVENTION AND PRIOR ART

The cytokine receptor CD137 is a member of the tumour necrosis factor receptor family. CD137 is expressed by activated T and B lymphocytes and expression in primary cells is strictly activation dependent (Schwarz et al., 1995). Soluble forms of CD137 are generated by differential splicing and are found at enhanced concentrations in sera of patients with rheumatoid arthritis (Michel et al., 1998). The gene for human CD137 resides on chromosome 1p36, in a cluster of related genes, and this chromosomal region is associated with mutations in several malignancies (Schwarz et al., 1997).

Crosslinking of CD137 costimulates proliferation of T lymphocytes (Goodwin et al., 1993; Pollock et al., 1993; Schwarz et al., 1996), and CD137 ligand expressed by B lymphocytes costimulates T cell proliferation synergistically with B7 (DeBenedette et al., 1995).

While agonistic antibodies and the ligand to CD137 enhance lymphocyte activation, CD137 protein has the opposite effect. It inhibits proliferation of activated T lymphocytes and induces programmed cell death. These T cell-inhibitory activities of CD137 require immobilisation of the protein, arguing for transmission of a signal through the ligand/coreceptor (Schwarz et al., 1996). The known human CD137 ligand is expressed constitutively by monocytes and its expression is inducible in T lymphocytes (Alderson et al., 1994). Monocytes are activated by immobilized CD137 protein and their survival is profoundly prolonged by CD137. (Langstein et al., 1998; Langstein et al., 1999a). CD137 also induces proliferation in peripheral monocytes (Langstein et al., 1999b). Macrophage colony-stimulating factor (M-CSF) is essential for the proliferative and survival-enhancing activities of CD137 (Langstein et al., 1999a; Langstein et al., 1999b).

WO99/44629, published Sep. 10, 1999, describes the use of CD137 in promoting the proliferation of peripheral monocytes. The application teaches that CD137, in particular in a multimerized or immobilized form, may be used to enhance growth and proliferation of peripheral monocytes. The application teaches further that CD137 induces proliferation of peripheral monocytes independently of hematopoietic stem cells (P. 4, line 25).

Cancer patients receiving a chemo- or/and radiation therapy suffer from a destruction or weakening of their immune and hematopoietic systems. The ability to reconstitute the immune and hematopoietic systems allows the application of higher doses of chemotherapy or/and radiation, increasing the chances of a complete removal of the tumor and metastases (Elias, 1995).

Sofar, granulocyte colony-stimulating factor (G-CSF) and granulocyte/macrophage colony-stimulating factor (GM-CSF) are used for reconstituting the immune and hematopoietic systems (Fan et al., 1991; Neidhart, 1992; Hofstra et al., 1996; Sachs, 1996). These factors are known to induce fever, chills, and other disagreeable and potentially dangerous symptoms in patients receiving them.

However, the use of such prior art methods of stimulating hematopoiesis rests upon the use of mainly a single cytokine, namely, G-CSF. It is evident that the natural process of hematopoiesis is not driven by such single factor alone, and hence, it is desirable to provide further factor(s), which may be used to that end. Furthermore, the use of G-CSF is hampered by the relatively short half-life of that substance, so that it is necessary to apply the factor repeatedly during the course of treatment. This is associated with patient discomfort as a number of intravenous applications are necessary. Still further, the response to G-CSF differs from patient to patient, and today it is not possible to foresee the response to the factor of a given patient. Therefore, in a number of cases too little G-CSF is applied, or the hematopoietic system does not rapidly enough or not to a large enough extent recover. The consequences of such insufficient response to the factor are an increased number of treatment-associated complications, such as opportunistic infection. Such infections can not always be brought under control, mainly because the patient's immune system is unable to successfully cope with them in the first place. Therefore, a number of patients may be severely harmed as a consequence of such infection, while others die of the infection.

Thus, it is necessary to discover further factors, which are useful in stimulating hematopoiesis by inducing proliferation and/or differentiation, in particular proliferation and/or differentiation of bone marrow and/or peripheral hematopoietic stem cells. These factors should advantageously feature a long-lasting, stimulating effect on the hematopoietic system, so that a sufficient impact on stimulation and/or regeneration of the hematopoietic system can be achieved.

Hematopietic stem cells, mesenchymal cells and multipotent adult progenitor cells contribute to the process of tissue repair and regeneration and wound healing (Jiang et al., 2002).

### SUMMARY OF THE INVENTION

The invention solves these prior art problems by providing CD137 molecule or a functional analogue thereof, for use in stimulating hematopoiesis. The invention further provides a method of treatment of a disorder characterized by insufficient numbers of cells of the hematopoietic system, including but not limited to T cells, B cells, erythroid cells, granulocytes, macrophages, mesenchymal cells, osteoclasts and multipotent adult progenitor cells comprising the application to a mammal, including a patient, in need thereof of an effective dose of CD137 or a functional analogue thereof.

The invention further comprises a method of treatment of a disorder characterized by an insufficient number of cells of the hematopoietic system, including but not limited to T cells, B cells, erythroid cells, granulocytes, erythrocytes, macrophages, mesenchymal cells, osteoclasts and multipotent adult progenitor cells comprising the application of CD137 or a functional analogue thereof to an isolated culture of stem cells or a mixed cell populations containing stem cells and the transfer of the treated cells to a mammal, including a patient, in need thereof.

The invention also comprises a method for the treatment of a mammal in need thereof wherein the mammal is characterized by having a decrease in the number or activity of its white or red blood cells of normal physiological function. Preferable, the decrease is caused by an immunodeficiency. Preferred immunodeficiencies include AIDS, hyperimmunoglobulin M syndrome, and other immunodeficiencies. Further preferably, the decrease is caused by and/or associated with chemo- and/or radiotherapy. The chemo- and/or radiotherapy is preferably administered to treat cancer.

Also preferably, the decrease is associated with leukopenia. The leukopenia is preferably caused by severe trauma, blood loss, immunodeficiency, or diseases such as agranulocytosis or bone marrow failure. Bone marrow failure may be due to congenital factors, toxins such as benzene, drug abuse, viral infections such as hepatitis, or side effects of immunotherapies.

Further preferably, the decrease is associated with a disease such as anemia, aplastic anemia, megablastic anemia, myelophthisic anemia, myelodysplastic syndrome or hairy cell leukemia.

Also preferably, the decrease is associated with a decrease in red blood cell number and/or function. The invention therefore provides a method of treatment for a mammal in need thereof wherein the mammal suffers from a disease or condition characterized by a decrease in the number and/or function of its red blood cells, comprising administering to the mammal an effective amount of CD137 or of a functional analog thereof, or comprising contacting erythroid cells or erythroid precursor cells with CD137 or a functional analog thereof, and administering the so-treated cells to the mammal. The cells may be derived from the mammal or from other sources. Preferably, they are derived from the mammal. The disorder or condition is preferably selected from anemia, anemia associated with renal failure or renal insufficiency, anemia of prematurity, severe antepartum iron deficiency anemia, postpartum anemia, and pernicious anemia.

The dose of the CD137 protein or functional analogue thereof of the invention is determined by the attending physician, or, in the case of a non-human mammal, by the researcher carrying out the experiment, or by a veterinarian. In general, effective doses of CD137 protein are within the range of 1 ng/kg to 1 mg/kg, more preferably 50 ng/kg to 100 μg/kg, most preferably 1 to 10 μg/kg.

The invention further provides a method for the stimulation of growth, proliferation, differentiation and/or activation of hematopoietic stem cells, comprising the step of contacting the cells with an effective amount of CD137, during a time period sufficient to allow for the said stimulation of growth, proliferation, differentiation and/or activation. The amount of CD137 is preferably between 1 to 10 μg/kg. The time period is preferably between 1 and 14 days.

The invention also provides the above method for the stimulation of growth, proliferation and/or differentiation of cells that are more differentiated that stem cells, such as erythroid cells which may mature into erythrocytes.

A further aspect of the invention is the provision of CD137 or a functional analogue thereof, for use in induction of proliferation of hematopoietic stem cells of a mammal. In another aspect, the invention provides CD137 or a functional analogue thereof, for use in stimulating hematopoiesis. In a further aspect, the invention provides CD137 or a functional analogue thereof, for use in tissue repair, tissue regeneration and wound healing. The invention also provides CD137 or a functional analogue thereof, for use in enhancing innate and/or adaptive immunity for cancer therapy. Still further, the invention provides CD137 or a functional analogue thereof, for use in enhancing innate and/or adaptive immunity for therapy of infectious disease. The invention also provides CD137 or a functional analogue thereof, for use in enhancing innate and/or adaptive immunity for vaccination against infectious disease. Also included in the scope of the invention is a method of treatment of a disorder characterized by insufficient numbers of cells of the hematopoietic system, including but not limited to T cells, B cells, granulocytes, macrophages, mesenchymal cells, osteoclasts and multipotent adult progenitor cells, comprising the step of administration to a mammal in need thereof of an effective dose of CD137 or a functional analogue thereof. The invention further comprises a method of treatment for a disorder characterized by an insufficient number of of cells of the hematopoietic system, including but not limited to T cells, B cells, granulocytes, macrophages, mesenchymal cells, osteoclasts and multipotent adult progenitor cells comprising the step of administration of CD137 or a functional analogue thereof to an isolated culture of stem cells and the transfer of the treated cells to a mammal in need thereof.

The invention also relates to a method for the treatment of a mammal in need thereof wherein the mammal is characterized by having a decrease in the number or activity of its white blood cells decrease is preferably caused by or associated with an immunodeficiency. The immunodeficiency is preferably selected from the group comprising AIDS, hyperimmunoglobulin M syndrome, radiation-induced immunodeficiency, and chemotherapy-induced immunodeficiency. The decrease is preferably associated with chemo- and/or radiotherapy and/or removal of blood progenitor cells. The chemo- and/or radiotherapy and/or removal of blood progenitor cells is preferably administered to treat cancer. The chemo- and/or radiotherapy and/or removal of blood progenitor cells is also preferably administered to treat autoimmune disease. Further preferably, the decrease is associated with leukopenia. The leukopenia is preferably caused by a condition selected from the group comprising severe trauma, blood loss, immunodeficiency, or disease such as agranulocytosis or bone marrow failure. The Bone marrow failure is preferably due to congenital factors, toxins such as benzene, street drugs, viral infections such as hepatitis, or side effects of immunotherapies. The leukopenia is preferably caused by a disease such as anemia, aplastic anemia, megablastic anemia, myelophthisic anemia, myelodysplastic syndrome or hairy cell leukemia.

The dose of CD137 protein or functional analog thereof administered is preferably within the range of 1 ng/kg to 1 mg/kg, more preferably 50 ng/kg to 500 μg/kg. Further preferably, the dose of CD137 protein or functional analog thereof administered is within the range 100 ng/kg to 100 μg/kg more preferably 500 ng/kg to 50 μg/kg. Still further preferably, the dose of CD137 protein or functional analog thereof administered is within the range of 1 to 10 μg/kg, more preferably 4 to 6 μg/kg.

The invention also comprises a method for the stimulation of growth, proliferation, differentiation and/or activation of hematopoietic stem cells, comprising the step of contacting the cells with an effective amount of CD137 or a functional analog thereof, during a time period sufficient to allow for said the stimulation of growth, proliferation, differentiation and/or activation. The concentration of CD137 is preferably from about 1 ng/ml to about 1 mg/ml, more preferably from about 5 ng/ml to about 400 mg/ml, further preferably from about 15 ng/ml to about 100 ng/ml, most preferably about 60 ng/ml. Similar concentration may be used with functional CD137 analogs, however. The person of skill in the art will appreciate that the concentrations of such analog may differ from the actual CD137 concentrations give here and that the optimum concentrations may be determined experimentally, for instance by determining the effective concentration of such analogs in functional assays such as the in vitro and in vivo assays described in the examples hereinbelow.

Therefore, invention also comprises a method as described above wherein the CD137 or functional analogue thereof is CD137, or a part of CD137, fused to Fc.

The CD137 used according to the invention preferably contains the extracellular part thereof. The CD137 preferably contains amino acids 18 to 255 thereof. Further preferably, the CD137 contains amino acids 18 to 186 thereof.

The invention also comprises the above methods wherein the CD137 or functional analogue thereof is used in combination with a growth factor.

The growth factor is preferably selected from among G-CSF, M-CSF, GM-CSF, IL-3, IFN-gamma, TNF, LIF, flt-3, and c-kit. More preferably, the growth factor is selected from among G-CSF, M-CSF and GM-CSF. Most preferably, the growth factor is G-CSF.

Preferably, in the methods of the invention, the CD137 or functional analogue thereof is administered as a single dose.

The invention also comprises a CD137 molecule, or functional analogue thereof, which is multimerized, for use according to the above embodiments or the above methods. The multimer preferably comprises 2 to 20 monomers, more preferably 3 to 10 monomers, still more preferably 3 to 5 monomers.

The monomers are preferably expressed a fusion protein. The monomers are also preferably fused together by means of a covalent bond.

The invention also comprises a molecule capable of crosslinking CD137 ligand(s) expressed on the surface of a target cell, for use according to the above embodiments of the invention.

The functional analog of CD137 is preferably an antibody or derived from an antibody. The functional analog is also preferably an anticalin or derived from an anticalin. Further preferably, the functional analog is a Trinectin or derived from a Trinectin. The functional analog is preferably characterized by the ability to stimulate the growth, proliferation, differentiation and/or activation of stem cells. The stem cells are hematopoietic stem cells. The stem cells are further preferably CD34 positive cells.

The invention also comprises a composition comprising CD137 or a functional analogue thereof, and a diluent and/or carrier. The composition of the invention is preferably suitable for dermal, transdermal, oral, intravenous, intraperitoneal, intramuscular, or intraliquoreal administration. The invention also provides a composition of the invention for use in the treatment of a disorder of a mammal wherein the disorder is associated with decreased number or activity of white blood cells. The decrease is preferably caused by or associated with an immunodeficiency. The immunodeficiency is preferably selected from the group comprising AIDS, hyperimmunoglobulin M syndrome, radiation-induced immunodeficiency, chemotherapy-induced immunodeficiency.

Within the scope of the invention is included a composition as described above for use in stimulation of proliferation and/or differentiation of hematopoietic stem cells. Such composition if preferably beneficial in disorders such as immunodeficiency, agranulocytosis, bone marrow failure, anemia, aplastic anemia, megablastic anemia, myelophthisic anemia, myelodysplastic syndrome or hairy cell leukemia.

The invention also comprises a method of treatment of a mammal wherein a therapeutically effective amount of a composition of the invention is administered to a mammal in need thereof, the mammal having a disorder associated with decreased proliferation and/or differentiation of stem cells.

The disorder is preferably cancer of cells of the hematopoietic lineage. The disorder is further preferably leukemia.

The invention also provides CD137 or a functional analogue thereof, for use in induction of proliferation of stem cells of a mammal. Also, The invention provides a method of treatment of a mammal in need thereof, wherein an effective dose of CD137 or a functional analog thereof is administered to the mammal, wherein the mammal suffers from a disorder associated with abnormal white blood cell number and/or function. The mammal preferably suffers from a disorder or condition selected from immunodeficiency, agranulocytosis, bone marrow failure, anemia, aplastic anemia, megablastic anemia, myelophthisic anemia, myelodysplastic syndrome or hairy cell leukemia and the disorder or condition is improved by the administration of CD137 or a functional analogue thereof.

The invention also provides inhibition of CD137 and/or inhibition of CD137Ligand signalling, for use in the treatment of a disorder or condition associated with excessive proliferation of hematopoietic cells.

The hematopoietic cells are preferably hematopoietic stem cells. The inhibition is preferably mediated by antibodies to CD137 or to CD137Ligand. These antibodies should be inhibitory to the binding of CD137 to CD137 Ligand, which may easily be determined by the skilled person, for instance by means of a binding assay where one of the partners is immobilized, for instance on a solid support such an an ELISA plate, and the other partner is provided in a soluble, labeled form. The binding can then be detected by assaying the presence of the label on the solid support, and likewise, the inhibition of such binding by the antibody may be ascertained by the absence or diminishing of label on the support in reactions where effective concentrations of such antibody have been added. Of course, the antibody may be tested in other ways, for instance by determining its inhibitory effect on the function of CD137 as measured in a functional assay as described hereinbelow in the examples. The skilled person will be aware that such antibody may by itself have a functional, stem cell proliferative, growth-enhancing and differentiation-stimulating activity like the CD137 molecules and functional analog described herein. Sich activity may be determined for instance in a functional assay as described in the examples hereinunder. Where such activity exists, and the antibody is to be used as inhibitor of the activation of cd137 Ligand through CD137, the preparation of Fab fragments or of single chain antibodies comprising a single binding domain only, will usually abolish such positive stimulatory activity, so that the resulting Fab fragment of single chain antibody may then be used as a inhibitor. Similarly to inhibitory antibodies, other agents may be used that are capable of inhibiting CD137 Ligand or CD137 expression. Such agents may be for instance antisense oligonucleotides, RNAi molecules, specific aptamers or aptazymes, which are capable of reducing transcription and/or expression of CD137 or CD137 Ligand. Such inhibition may then result in lowered stimulation of proliferation through the CD137/CD137 Ligand pathway, which in turn is desirable in disorders where cells of the hematopoietic lineage proliferate out of control. Such disorders include stem cell leukemia and myeloproliferative disease.

Another use of the CD137 molecules of the invention is the provision of the coding sequence therefor, in particular, of the coding sequence of the extracellular domain thereof, preferably amino acids 18 to 255 thereof, more preferably amino acids 18 to 186 thereof. This coding sequence is preferably provided in fused to transcriptional control elements such as a promoter and optionally, an enhancer. The coding sequence is further preferably fused to a transcription terminator and/or a Polyadenylation sequence. The promoter is preferably inducible, and also preferably tissue-specific. For instance, promoters specific to white blood cells such as T cells, B cells, Monocytes, Granulocytes, and the like are preferred. The coding sequence preferably comprises or is fused to a leader sequence, such as the leader sequence of CD137. The coding sequence may be provided as a multimer, such that one coding sequence is fused to at least one more coding seuqence. The coding seuqences are preferably not fised together directly, but sepoarated by a spacer sequence. The spacer sequence is preferably coding for one to 50 amino acids, more preferably for 5 to 30, most preferably for 9 to 25 amino acids. The spacer sequence may for instance be derived from the sequence coding for the hinge region of human antibodies. The coding sequence may thus be introduced into a cell which is then able to produce and secrete the CD137 protein or fragment or multimer thereof. Thus, the so-produced CD137 fragment or multimer thereof may be administered to a mammal in need thereof, according to the above embodiments of the invention. Alternatively, the cell comprising the coding sequence may be introduced into the mammal, providing the mammal though the CD137 produced by such cell internally with the Cd137 molecules of the invention. Still further, the coding sequence may be introduced into a mammal, either as naked DNA (for methods see e.g., U.S. Pat. Nos. 5,580,859, 5,589,466, and 5,693,622), or through lipid- or liposome-mediated DNA uptake methods (see e.g., U.S. Pat. Nos. 5,194,654, 5,223,263, 5,264,618, 5,459,127, and 5,703,055), or through methods where DNA uptake is mediated by other chemicals (see e.g., U.S. Pat. No. 6,022,874). Also methods using retroviral- or other biological methods are well known to the person of skill in the art.

The CD137 protein as a soluble protein will generally be incative as concerns the stimulation of proliferation, growth and/or differentiation of stem cells. Therefore, in order to achieve the acitvity of the CD137 protein of the invention, it is generally necessary to multimerize the protein, either by linking or fusing CD137 monomers, or by expressing the CD137 protein on the surface of cells, or by introducing it to liposomes or the like bodies which are shaped similarly to cells, or by immobilizing the CD137 protein on the surface of beads, or by immobilizing it by using agents such as protein A, as described hereinbelow. Similar considerations apply to functional analog of CD137, which may have to be multimerized as well in order to become functional within the meaning of the invention.

Thus, the CD137 protein is preferably multimerized, preferably in multimers of 2-20 molecules, more preferably in multimers of at least 3. Multimerization may be achieved by a variety of means. For instance, CD137 molecules may be coupled by chemical cross-linking. It is important not to destroy the biological activity of CD137 molecules crosslinked in this manner. A number of cross-linkers have been developed, some of which comprise spacer molecules in order to prevent steric hindrance to binding or other biological activities. As cross-linker, the following molecules may be advantageously used:

Examples of homobifunctional crosslinkers include:
sulfosuccinimidyl 4-[N-maleimidomethyl]-cyclohexane-1-carboxylate (sulfo-SMCC) (see e.g., Z. Liu et al., J. Immunol. Methods 234(1-2): P153-67, 2000),
3,3′-dithio bis(sulfosuccinimidylpropionate),
BMME (Weston et al., Biochem Biophys. Acta 612, 40, 1980)
BSOCOES (Howard et al., J. Biol. Chem. 260, 10833, 1985),
DSP (Lee and Conrad, J. Immunol. 124, 518, 1985),
DSS D'Souza et al., J. Biol. Chem. 263, 3943, 1988),
EGS, optionally water-soluble (Geisler et al., Eur. J. Biochem. 206, 841, 1992, Moenner et al., PNAS 83, 5024, 1986, Yanagi et al., Agric Biol. Chem. 53, 525, 1989),
SATA (Duncan et al., Anal. Biocehm. 132, 68, 1983).

Examples of heterobifunctional crosslinkers include:
GMBS (Kitagwa et al., J. Biochem. 94, 1160, 1983, Rusin et al.,
Biosens. Bioelectron. 7, 367, 1992),
MBS (Green et al., Cess 28, 477, 1982),
PMPI (Aithal et al., J. Immunol. Meth. 112, 63, 1988),
SMCC (Annunziato et al., Bioconjugate Chem. 4, 212, 1993),
SPDP (Caruelle et al., Anal. Biochem. 173, 328).

Crosslinkers are available commercially e.g., from Calbiochem, 10394 Pacific Center Court, San Diego, Calif. 92121, USA, Pierce Chemicals 4722 Bronze way, Dallas, Tex., 75236, USA, Dalton Chemical Laboratories Inc. 349 Wildcat Rd., Toronto, ON, M3J 2S3, Canada.

Cross-linking of CD137 molecules may also be achieved through linking the molecules by using a peptide linker, or by attaching two or more CD137 molecules to another protein. Such molecules may be expressed using recombinant molecular DNA techniques. Another possibility is coupling the CD137 molecule to a protein that is known to dimerize or multimerize. Such proteins will, upon expression, dimerize or multimerize, so that the CD137 moiety fused thereto will be dimerized or multimerized as well. An example of this technology is the TNF-R-Fc fusion protein constructed by Peppel et al., J. Exp. Med. 174:1483-9, 1991. A molecule constructed in that manner, Etanercept or Enbrel, is now manufactured by Amgen and used for treatment of rheumatoid arthritis.

The biological activity of the cross-linked molecule obtained in this manner may be assayed by any assay wherein the CD137 molecule or a functional analogue thereof is tested for its ability to stimulate the proliferation, growth, differentiation and/or activation of hematopoietic stem cells. Such assays may comprise stem cells, preferably peripheral stem cells, isolated from a mammal, preferably a human. The stem cells are preferably CD34 positive peripheral cells. Such cells may be isolated in ways known to the person of skill in the art, for instance, by panning on a surface covered with anti-CD34 antibodies, by FACSort using labeled anti-CD34 antibodies, by isolation using anti-CD34 antibodies bound to magnetic beads, or the like techniques. An example of a test for stem cell proliferation is described in example 1 hereunder.

The CD137 molecules of the invention preferably comprise the amino acid sequence of the native CD137 molecule, i.e., amino acids 18 to 255. More preferable, the CD137 molecule of the invention comprises the soluble, extracellular part of the CD137 molecule, i.e., amino acids 18 to 186. Of course, functional analogues of such molecules may be used as well. The transmembrane domain of CD137 (amino acids 18 to 186) may advantageously be included when it is desired to multimerize the CD137 molecule through lipophilic affinities, for instance, in a liposome or when expressing the CD137 molecule on the surface of a cell.

Without wishing to be bound by theory, it is the inventor's belief that the effect of CD137 described herein is mediated by the cross-linking of CD137 ligands on the cells that are so effected, preferably stem cells Therefore, according to the invention any molecule may serve as a functional analogue of CD137 within the context of this description, that is able to cross-link CD137 ligand(s) on target cells and in that manner causes stimulation of growth, proliferation, differentiation and/or activation of hematopoietic stem cells. Functional analogues of CD137 may be found by deleting amino acid sequences from the sequence of the native CD137 that are not involved in binding CD137 ligand or whose deletion does not interfere substantially with binding. The biological effects of such molecules may be tested as mentioned hereinabove, in particular as described in example 1. Further, it is possible to exchange amino acids within the sequence of CD137 with other amino acids, preferably with similar amino acids, as described in more detail below. Still further, naturally occurring analogues and/or homologues of CD137 may be used and tested for their activity on the desired mammalian stem cells which are preferably human. Such homologs include the murine CD137 homolog, 4-1BB (Kwon and Weissman, 1989). Further, anti-CD137 Ligand antibodies, or fragments thereof having the ability to cross-link CD137 molecules which are located on the surface of a cell membrane, may be used in place of CD137. Also other members of the TNR-R family may be used, provided they (1) are able to cross-link CD137 molecules that are located on a cell surface membrane, or (2) they exert the actions of CD137 described herein, that is, activation, proliferation, differentiation and/or growth.

The functional analogues of the CD137 molecule according to the invention also comprise molecules that are not derived from CD137 sequence. For instance, antibodies against CD137 ligand(s) may be used. The activity of such antibodies may be further enhanced by cross-linking the antibodies, e.g. using antibodies directed against the Fc part of the CD137 ligand(s)-binding antibodies. For instance, mouse anti rabbit-antibodies could be used when the CD137 ligand(s) binding antibody is a rabbit antibody.

Of course, the antigen-binding part of such antibodies may be used alone (e.g., as single-chain antibody) and multimerized in a like manner as detailed above for CD137 protein. Multimerization of such antibodies may be used to enhance the potency thereof.

Furthermore, the functional analogue may be selected from among molecules that, like antibodies, specifically bind to CD137 ligand(s) and, when such molecules are used in a multimerized form, are able to cross-link CD137 ligand. Such compounds can e.g. be antibodies, small molecules, recombinant phages, or peptides. Suitable molecules are e.g., anticalins, described in EP1017814. Said European patent also describes the process of preparing such anticalins with the ability to bind a specific target. Further suitable molecules are Trinectins (Phylos Inc., Lexington, Mass., USA, and Xu et al., Chem. Biol. 9:933, 2002). Another kind of suitable molecule are affybodies (see Hansson et al., Immunotechnology 4(3-4):237-52, 1999, and Henning et al., Hum Gene Ther. 13(12):1427-39, 2002, and references therein) The activity of CD137, fragments thereof, mutations thereof, cross-linked variants thereof, or generally of functional analogues thereof and of any variations and changes to these molecules, within the context of the invention, may be ascertained by the skilled person by assaying for (1) binding of such analogues, fragments, variants, etc. to CD137 ligand(s), or (2) by assaying the biological function of CD137 as described hereinabove, namely, the stimulation of growth, proliferation, differentiation and/or activation of stem cells, preferably human or murine stem cells, more preferably hematopoietic stem cells. Assays for the detection and measurement of growth, proliferation, differentiation or activation are well known to the skilled person. For instance, growth may simply be determined by counting the cell number, subjecting the cells to the conditions of the assay, for instance by incubating the cells in the presence and in the absence of a molecule of the invention, and recounting the number of cells after a suitable period of time, which will generally be 1-14 days, preferably 2-10 days. Further by way of illustration, the procedures described in the examples, for instance, in example 1, and in examples 2 and 3, hereinbelow, may be used to assay cell proliferation. Activation of stem cells usually is accompanied by activation of extracellular stress-regulated kinase (ERK1/2), ribosomal S6 kinase (p90RSK), Akt and the mitogen activated protein kinase (MAPK) pathway (see e.g., Lee et al., Blood. 99(12):4307-17, 2002; Rozenfeld-Granot et al., Exp Hematol. 30(5):473-80, 2002). Thus, the skilled artisan may determine for each of the molecules of the invention whether it is capable of activating, stimulating growth and/or stimulating proliferation, and/or stimulating differentiation of stem cells, by using hematopoietic stem cells or other stem cells, derived from peripheral cells or from bone marrow. The stem cells may be human or other stem cells. Preferably are murine or human stem cells. Further preferred are bone marrow derived stem cells. Also preferred are hematopoietic stem cells. Most preferred are human bone-marrow derived hematopoietic stem cells. Also preferred are human peripheral hematopoietic stem cells. CD34 positive cells are the preferred source of stem cells.

The CD137 molecules and functional analogues thereof which are contemplated within the scope of the invention are useful for the stimulation of growth, proliferation, differentiation and/or activation of stem cells. Thus, these molecules are useful in the treatment of disorder where such stem cells lack activation, or where proliferation or where differentiation and/or growth thereof is beneficial. Such disorders and conditions include immunodeficiencies, including those that are associated with treatment with radiation therapy and/or chemotherapy. As such, the molecules of the invention may not only be administered to humans, but also to other mammals, including but not limited to dogs, cats, guinea pigs, hamsters, horses, rabbits, goats, sheep, cows, oxen, bulls, pigs, mice and rats.

Further, the molecules of the invention may be useful in enhancing the number of stem cells in poultry, including chicken and turkey.

The molecules of the invention may be used ex vivo, in order to stimulate growth, activation, differentiation and/or proliferation of stem cells in a culture of such cells, or in vivo. For in vivo use, compositions need to be prepared that are compatible with their administration to a patient or non-human animal. Compositions that are injected usually comprise the active ingredient together with a pharmaceutically acceptable buffer and/or diluent. For dermal application, the active ingredient, which is usually a protein or peptide, may be modified using the technique described by Foldvari et al., U.S. Pat. No. 6,444,200, included herein by reference. According to said Foldvari et al., a protein or peptide may be coupled to at least one unsaturated fatty acid moiety having between 16-20 carbon atoms, which is covalently attached to the protein or peptide. The said fatty acid is preferably oleic acid. Further to enhance penetration of dermis, the methods described further hereinbelow for conferring on peptides the ability to cross membranes, may also be used. Pharmaceutical compositions and preparations may comprise pharmaceutically acceptable carriers and/or diluents. Pharmaceutically acceptable carriers or diluents include those used in formulations suitable for oral or parenteral (e.g. intramuscular or intravenous) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product. For example, formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the polypeptide to blood components or one or more organs. Suitable liposomes include, for example, those comprising the positively charged lipid (N[1-(2,3-dioleyloxy)propyl]-N,N,N-triethylammonium (DOTMA), those comprising dioleoylphosphatidylethanolamine (DOPE), and those comprising 3beta[N-(n′,N′-dimethylaminoethane)-carbamoyl]cholesterol (DC-Chol). Optionally, the proteins, peptides and/or other molecules of the invention may be attached to drug delivery substrates in order to facilitate their delivery to the correct location in the mammal, preferably the patient. Suitable substrates include polymeric beads known in the art. Such beads may, preferably, be engineered to target the peptides of the invention to a given location in the body.

The present invention also concerns the DNA sequence encoding a CD137 protein and the CD137 proteins encoded by the DNA sequences. Such DNA sequences may be used according to the invention to express proteins or peptides having the function of the CD137 proteins of the invention, i.e., stimulation of growth, proliferation, differentiation and/or activation of hematopoietic stem cells.

Moreover, as mentioned hereinabove, the present invention further concerns the DNA sequences encoding biologically active analogs, fragments and derivatives of the CD137 protein, and the analogs, fragments and derivatives encoded thereby. The preparation of such analogs, fragments and derivatives is by standard procedure (see for example, Sambrook et al., 1989) in which in the DNA sequences encoding the CD137 protein, one or more codons may be deleted, added or substituted by another, to yield analogs having at least one amino acid residue change with respect to the native protein.

Of the above DNA sequences of the invention which encode a CD137 protein, isoform, analog, fragment or derivative, there is also included, as an embodiment of the invention, DNA sequences capable of hybridizing with a cDNA sequence derived from the coding region of a native CD137 protein, in which such hybridization is performed under moderately stringent conditions, or preferably, under more stringent, or most preferably, under stringent conditions, and which hybridizable DNA sequences encode a biologically active CD137 protein. These hybridizable DNA sequences therefore include DNA sequences which have a relatively high homology to the native CD137 cDNA sequence and as such represent CD137-like sequences which may be, for example, naturally-derived sequences encoding the various CD137 isoforms, or naturally-occurring sequences encoding proteins belonging to a group of CD137-like sequences encoding a protein having the activity of CD137. Further, these sequences may also, for example, include non-naturally occurring, synthetically produced sequences, that are similar to the native CD137 cDNA sequence but incorporate a number of desired modifications. Such synthetic sequences therefore include all of the possible sequences encoding analogs, fragments and derivatives of CD137, all of which have the activity of CD137.

To obtain the various above noted naturally occurring CD137-like sequences, standard procedures of screening and isolation of naturally-derived DNA or RNA samples from various cell types of mammals, preferably cells derived from hematopoietic precursors or such precursors, may be employed using the natural CD137 cDNA or portion thereof as probe (see for example standard procedures set forth in Sambrook et al., 1989).

Likewise, to prepare the above noted various synthetic CD137-like sequences encoding analogs, fragments or derivatives of CD137, a number of standard procedures may be used as are detailed herein below concerning the preparation of such analogs, fragments and derivatives.

A polypeptide or protein “substantially corresponding” to CD137 protein includes not only CD137 protein but also polypeptides or proteins that are analogs of CD137.

Analogs that substantially correspond to CD137 protein are those polypeptides in which one or more amino acid of the CD137 protein's amino acid sequence has been replaced with another amino acid, deleted and/or inserted, provided that the resulting protein exhibits substantially the same or higher biological activity as the CD137 protein to which it corresponds.

In order to substantially correspond to CD137 protein, the changes in the sequence of CD137 proteins, such as isoforms are generally relatively minor. Although the number of changes may be more than fifty, they are preferably no more than about thirty, still more preferably about ten, more preferably no more than five, and most preferably no more than three such changes. While any technique can be used to find potentially biologically active proteins, which substantially correspond to CD137 proteins, one such technique is the use of conventional mutagenesis techniques on the DNA encoding the protein, resulting in a few modifications. The proteins expressed by such clones can then be screened for their activity in inducing stem cell proliferation, growth, differentiation and/or activation, for instance by using the assays described hereinbelow, e.g., in example 1, and in Examples 2 and 3 hereinbelow.

“Conservative” changes are those changes which would not be expected to change the activity of the protein and are usually the first to be screened as these would not be expected to substantially change the size, charge or configuration of the protein and thus would not be expected to change the biological properties thereof.

Conservative substitutions of CD137 proteins include an analog wherein at least one amino acid residue in the polypeptide has been conservatively replaced by a different amino acid. Such substitutions preferably are made in accordance with the following list as presented in Table A, which substitutions may be determined by routine experimentation to provide modified structural and functional properties of a synthesized polypeptide molecule while maintaining the biological activity characteristic of CD137 protein.

TABLE A

| Original | Exemplary |
|---|---|
| Residue | Substitution |
| Ala | Gly; Ser |
| Arg | Lys |
| Asn | Gln; His |
| Asp | Glu |
| Cys | Ser |
| Gln | Asn |
| Glu | Asp |
| Gly | Ala; Pro |
| His | Asn; Gln |
| Ile | Leu; Val |
| Leu | Ile; Val |
| Lys | Arg; Gln; Glu |
| Met | Leu; Tyr; Ile |
| Phe | Met; Leu; Tyr |
| Ser | Thr |
| Thr | Ser |
| Trp | Tyr |
| Tyr | Trp; Phe |
| Val | Ile; Leu |

Alternatively, another group of substitutions of CD137 protein are those in which at least one amino acid residue in the polypeptide has been removed and a different residue inserted in its place according to the following Table B. The types of substitutions which may be made in the polypeptide may be based on analysis of the frequencies of amino acid changes between a homologous protein of different species, such as those presented in Table 1-2 of Schulz et al., G. E., Principles of Protein Structure Springer-Verlag, New York, N.Y., 1798, and FIGS. 3-9 of Creighton, T. E., Proteins: Structure and Molecular Properties, W.H. Freeman & Co., San Francisco, Calif. 1983. Based on such an analysis, alternative conservative substitutions are defined herein as exchanges within one of the following five groups:

TABLE B

| | |
|---|---|
| 1. | Small aliphatic, nonpolar or slightly polar residues: Ala, Ser, |
| | Thr (Pro, Gly); |
| 2. | Polar negatively charged residues and their amides: Asp, Asn, |
| | Glu, Gln; |
| 3. | Polar, positively charged residues: |
| | His, Arg, Lys; |
| 4. | Large aliphatic nonpolar residues: |
| | Met, Leu, Ile, Val (Cys); and |
| 5. | Large aromatic residues: Phe, Tyr, Trp. |

The three amino acid residues in parentheses above have special roles in protein architecture. Gly is the only residue lacking any side chain and thus imparts flexibility to the chain. This however tends to promote the formation of secondary structure other than a-helical. Pro, because of its unusual geometry, tightly constrains the chain and generally tends to promote β-turn-like structures, although in some cases Cys can be capable of participating in disulfide bond formation which is important in protein folding. Note that Schulz et al., supra, would merge Groups 1 and 2, above. Note also that Tyr, because of its hydrogen bonding potential, has significant kinship with Ser, and Thr, etc.

Conservative amino acid substitutions according to the present invention, e.g., as presented above, are known in the art and would be expected to maintain biological and structural properties of the polypeptide after amino acid substitution. Most deletions and substitutions according to the present invention are those which do not produce radical changes in the characteristics of the protein or polypeptide molecule. “Characteristics” is defined in a non-inclusive manner to define both changes in secondary structure, e.g. α-helix or β-sheet, as well as changes in biological activity, e.g., stimulation of growth, proliferation, differentiation and/or activation of stem cells, and/or cross-linking of CD137 ligand(s), preferably CD137 ligand(s) expressed on stem cells.

Examples of production of amino acid substitutions in proteins which can be used for obtaining analogs of CD137 proteins for use in the present invention include any known method steps, such as presented in U.S. patent RE 33,653, 4,959,314, 4,588,585 and 4,737,462, to Mark et al.; 5,116,943 to Koths et al., 4,965,195 to Namen et al.; 4,879,111 to Chong et al.; and 5,017,691 to Lee et al.; and lysine substituted proteins presented in U.S. Pat. No. 4,904,584 (Shaw et al.).

Besides conservative substitutions discussed above which would not significantly change the activity of CD137 protein, either conservative substitutions or less conservative and more random changes, which lead to an increase in biological activity of the analogs of CD137 proteins, are intended to be within the scope of the invention.

When the exact effect of the substitution or deletion is to be confirmed, one skilled in the art will appreciate that the effect of the substitution(s), deletion(s), etc., will be evaluated by routine binding, proliferation, growth, differentiation and/or activation assays, preferably using hematopoietic stem cells as target cells. Screening using such a standard test does not involve undue experimentation.

Acceptable CD137 analogs are those which retain at least the capability of binding to CD137 ligand(s), and thereby, as noted above when used as in a multimerized form, are able to cross-link said CD137 ligand(s).

At the genetic level, these analogs are generally prepared by site-directed mutagenesis of nucleotides in the DNA encoding the CD137 protein, thereby producing DNA encoding the analog, and thereafter synthesizing the DNA and expressing the polypeptide in recombinant cell culture. The analogs typically exhibit the same or increased qualitative biological activity as the naturally occurring protein, Ausubel et al., Current Protocols in Molecular Biology, Greene Publications and Wiley Interscience, New York, N.Y., 1987-1995; Sambrook et al., Molecular Cloning: A Laboratory Manual, Cold Spring Harbor Laboratory, Cold Spring Harbor, N.Y., 1989.

Preparation of a CD137 protein in accordance herewith, or an alternative nucleotide sequence encoding the same polypeptide but differing from the natural sequence due to changes permitted by the known degeneracy of the genetic code, can be achieved by site-specific mutagenesis of DNA that encodes an earlier prepared analog or a native version of a CD137 protein. Site-specific mutagenesis allows the production of analogs through the use of specific oligonucleotide sequences that encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 20 to 25 nucleotides in length is preferred, with about 5 to 10 complementing nucleotides on each side of the sequence being altered. In general, the technique of site-specific mutagenesis is well known in the art, as exemplified by publications such as Adelman et al., DNA 2:183 (1983), or Xu et al., Biotechniques 32:1266-8, 2002 (and references therein), the disclosure of which is incorporated herein by reference.

As will be appreciated, the site-specific mutagenesis technique typically employs a phage vector that exists in both a single-stranded and double-stranded form. Typical vectors useful in site-directed mutagenesis include vectors such as the M13 phage, for example, as disclosed by Messing et al., Third Cleveland Symposium on Macromolecules and Recombinant DNA, Editor A. Walton, Elsevier, Amsterdam (1981), the disclosure of which is incorporated herein by reference. These phage are readily available commercially and their use is generally well known to those skilled in the art. Alternatively, plasmid vectors that contain a single-stranded phage origin of replication (Veira et al., Meth. Enzymol. 153:3, 1987) may be employed to obtain single-stranded DNA.

In general, site-directed mutagenesis in accordance herewith is performed by first obtaining a single-stranded vector that includes within its sequence a DNA sequence that encodes the relevant polypeptide. An oligonucleotide primer bearing the desired mutated sequence is prepared synthetically by automated DNA/oligonucleotide synthesis. This primer is then annealed with the single-stranded protein-sequence-encoding vector, and subjected to DNA-polymerizing enzymes such as E. coli polymerase I Klenow fragment, to complete the synthesis of the mutation-bearing strand. Thus, a mutated sequence and the second strand bear the desired mutation. This heteroduplex vector is then used to transform appropriate cells, such as E. coli JM101 or XL-1 Blue cells, and clones are selected that include recombinant vectors bearing the mutated sequence arrangement.

After such a clone is selected, the mutated CD137 protein sequence may be removed and placed in an appropriate vector, generally a transfer or expression vector of the type that may be employed for transfection of an appropriate host.

Accordingly, gene or nucleic acid encoding for a CD137 protein can also be detected, obtained and/or modified, in vitro, in situ and/or in vivo, by the use of known DNA or RNA amplification techniques, such as PCR and chemical oligonucleotide synthesis. PCR allows for the amplification of specific DNA sequences by repeated DNA polymerase reactions. This reaction can be used as a replacement for cloning; all that is required is a knowledge of the nucleic acid sequence. In order to carry out PCR, primers are designed which are complementary to the sequence of interest. The primers are then generated by automated DNA synthesis. Because primers can be designed to hybridize to any part of the gene, conditions can be created such that mismatches in complementary base pairing can be tolerated. Amplification of these mismatched regions can lead to the synthesis of a mutagenized product resulting in the generation of a peptide with new properties (i.e., site directed mutagenesis). See also, e.g., Ausubel, supra, Ch. 16. Also, by coupling complementary DNA (cDNA) synthesis, using reverse transcriptase, with PCR, RNA can be used as the starting material for the synthesis of the CD137, or of a preferably functional part thereof, without cloning.

Furthermore, PCR primers can be designed to incorporate new restriction sites or other features such as termination codons at the ends of the gene segment to be amplified. This placement of restriction sites at the 5′ and 3′ ends of the amplified gene sequence allows for gene segments encoding CD137 protein or a fragment thereof to be custom designed for ligation other sequences and/or cloning sites in vectors.

PCR and other methods of amplification of RNA and/or DNA are well known in the art and can be used according to the present invention without undue experimentation, based on the teaching and guidance presented herein. Known methods of DNA or RNA amplification include, but are not limited to polymerase chain reaction (PCR) and related amplification processes (see, e.g., U.S. Pat. Nos. 4,683,195, 4,683,202, 4,800,159, 4,965,188, to Mullis et al.; U.S. Pat. Nos. 4,795,699 and 4,921,794 to Tabor et al.; U.S. Pat. No. 5,142,033 to Innis; U.S. Pat. No. 5,122,464 to Wilson et al.; U.S. Pat. No. 5,091,310 to Innis; U.S. Pat. No. 5,066,584 to Gyllensten et al.; U.S. Pat. No. 4,889,818 to Gelfand et al.; U.S. Pat. No. 4,994,370 to Silver et al.; U.S. Pat. No. 4,766,067 to Biswas; U.S. Pat. No. 4,656,134 to Ringold; and Innis et al., eds., PCR Protocols: A Guide to Method and Applications) and RNA mediated amplification which uses anti-sense RNA to the target sequence as a template for double stranded DNA synthesis (U.S. Pat. No. 5,130,238 to Malek et al., with the tradename NASBA); and immuno-PCR which combines the use of DNA amplification with antibody labeling (Ruzicka et al., Science 260:487 (1993); Sano et al., Science 258:120 (1992); Sano et al., Biotechniques 9:1378 (1991)), the entire contents of which patents and reference are entirely incorporated herein by reference.

Similarly, derivatives may be prepared by standard modifications of the side groups of one or more amino acid residues of the CD137 protein, its analogs or fragments, or by conjugation of the CD137 protein, its analogs or fragments, to another molecule e.g. an antibody, enzyme, receptor, etc., as are well known in the art. Accordingly, “derivatives” as used herein covers derivatives which may be prepared from the functional groups which occur as side chains on the residues or the N- or C-terminal groups, by means known in the art, and are included in the invention. Derivatives may have chemical moieties such as carbohydrate or phosphate residues, provided such a fraction has the same or higher biological activity as CD137 proteins.

For example, derivatives may include aliphatic esters of the carboxyl groups, amides of the carboxyl groups by reaction with ammonia or with primary or secondary amines, N-acyl derivatives or free amino groups of the amino acid residues formed with acyl moieties (e.g., alkanoyl or carbocyclic aroyl groups) or O-acyl derivatives of free hydroxyl group (for example that of seryl or threonyl residues) formed with acyl moieties.

The term “derivatives” is intended to include only those derivatives that do not change one amino acid to another of the twenty commonly occurring natural amino acids.

CD137 is a protein or polypeptide, i.e. a sequence of amino acid residues. A polypeptide consisting of a larger sequence which includes the entire sequence of a CD137 protein, in accordance with the definitions herein, is intended to be included within the scope of such a polypeptide as long as the additions do not affect the basic and novel characteristics of the invention, i.e., if they either retain or increase the biological activity of CD137 protein or can be cleaved to leave a protein or polypeptide having the biological activity of CD137 protein. Thus, for example, the present invention is intended to include fusion proteins of CD137 protein or part of the CD137 protein with other amino acids or peptides, for instance as described hereinabove.

The CD137 protein, their analogs, fragments and derivatives thereof, have a number of possible uses, for example:

CD137 protein, its analogs, fragments and derivatives thereof, may be used to enhance the function of naturally-occurring CD137 in mammals, specifically, in stem cells and/or in cells of the hematopoietic system and/or lineage. For example, if CD137 is expressed on the surface of a cell, which comes in contact with stem cells, then introducing the CD137 gene into such cells of a mammal can enhance hematopoiesis by stimulating the proliferation, growth, differentiation and/or activation of stem cells. In this case the CD137 protein, its analogs, fragments or derivatives thereof, which have the desired stem cell stimulating effect, may be introduced to the cells by standard procedures known per se. It is possible to introduce the CD137 gene as described further above, or as illustrated in the examples below. Another possibility is to introduce the sequences of the CD137 protein (e.g., any one of the CD137 or its isoforms) in the form of oligonucleotides which can be absorbed by the cells and expressed therein.

Another possibility is to use antibodies specific for the CD137 protein to inhibit its stem cell stimulating effects. This may be desirable where the immune system of a patient is overly active, e.g., in cases of autoimmune diseases such as arteriosclerosis, arthritis, Crohn's disease, Hashimoto's thyroiditis, Addison's disease, juvenile diabetes, diabetes, rheumatoid arthritis, systemic lupus erythematosus, dermatomyositis, Sjogren's syndrome, dermatomyositis, lupus erythematosus, multiple sclerosis, myasthenia gravis, Reiter's syndrome, Graves disease, systemic lupus erythematosus, ulcerative colitis, psoriasis, multiple sclerosis, myasthenia gravis, and other diseases caused by or associated with autoimmunity, or in cancers arising from the hematopoietic system, such as granulomas, myeloproliferative disease, stem cell leukemia, other leukemias and generally all types of malignant growth associated with excess growth of cells derived from the heamtopoietic system.

One way of inhibiting the CD137 protein is by a ribozyme approach. Ribozymes are catalytic RNA molecules that specifically cleave RNAs. Ribozymes may be engineered to cleave target RNAs of choice, e.g., the mRNAs encoding the CD137 protein of the invention. Such ribozymes would have a sequence specific for the CD137 protein mRNA and would be capable of interacting therewith (complementary binding) followed by cleavage of the mRNA, resulting in a decrease (or complete loss) in the expression of the CD137 protein, the level of decreased expression being dependent upon the level of ribozyme expression in the target cell. To introduce ribozymes into the cells of choice, any suitable vector may be used, e.g., plasmid, bacterial vectors, that are usually used for this purpose (see also (i) above, where the vector has, as second sequence, a cDNA encoding the ribozyme sequence of choice). (For reviews, methods etc. concerning ribozymes see Chen et al., 1992; Zhao and Pick, 1993; Shore et al., 1993; Joseph and Burke, 1993; Shimayama et al., 1993; Cantor et al., 1993; Barinaga, 1993; Crisell et al., 1993 and Koizumi et al., 1993).

Further, CD137 expression may be inhibited by using antisense oligodeoxynucleotides and/or RNA interference through small interfering RNAs or small hairpin RNAs (see e.g., Hannon, Nature 418(6894):244-51, 2002, Ueda, J. Neurogenet. 15(3-4):193-204, 2001, Lindenbach and Rice, Mol. Cell. 9(5):925-7, 2002, Brantl, Biochim Biophys Acta. 1575(1-3):15-25, 2002.

As noted hereinabove and hereinbelow, the CD137 protein, or its analogs, fragments or derivatives thereof, of the invention may also be used as immunogens (antigens) to produce specific antibodies thereto. These antibodies may also be used for the purposes of purification of the CD137 protein (e.g., CD137 or any of its isoforms) either from cell extracts or from transformed cell lines producing CD137 protein, or its analogs or fragments.

It should also be noted that the isolation, identification and characterization of the CD137 protein of the invention may be performed using any of the well known standard screening procedures. As noted above and below, procedures may be employed such as affinity chromatography, phage display, DNA hybridization procedures, etc. as are well known in the art, to isolate, identify and characterize the CD137 protein of the invention or to isolate, identify and characterize additional proteins, factors, etc. which are capable of binding to the CD137 ligand and/or to stimulate the proliferation, growth, differentiation and/or activation of stem cells.

As set forth hereinabove, the CD137 protein may be used to generate antibodies specific to CD137 proteins, e.g., CD137 and its isoforms. These antibodies or fragments thereof may be used as set forth hereinbelow in detail, it being understood that in these applications the antibodies or fragments thereof are those specific for CD137 proteins.

Since it may be advantageous to design peptide inhibitors that selectively inhibit CD137 activity without interfering with other physiological processes in which other proteins are involved, the pool of peptides binding to CD137 in an assay such as the one described by Geysen (Geysen, 1985; Geysen et al., 1987) can be further synthesized as a fluorogenic peptide to test for selective binding to such other proteins to select only those specific for CD137 Such peptides may then further investigated for inhibition of the binding of CD137 to its ligand(s). Peptides which are able to inhibit such interaction may then be selected as candidate structures for molecules that are able to inhibit the activity of CD137 described hereinbelow and hereinabove. Peptides which are determined to be specific can then be modified to enhance cell permeability and inhibit the activity of CD137 either reversibly or irreversibly. Accordingly, peptides that selectively bind to CD137 can be modified with, for example, an aldehyde group, chloromethylketone, (acyloxy)methyl ketone or a CH₂OC (O)-DCB group to create a peptide inhibitor of CD137 activity. Further, to improve permeability, peptides can be, for example, chemically modified or derivatized to enhance their permeability across the cell membrane and facilitate the transport of such peptides through the membrane and into the cytoplasm. Muranishi et al. (1991) reported derivatizing thyrotropin-releasing hormone with lauric acid to form a lipophilic lauroyl derivative with good penetration characteristics across cell membranes. Zacharia et al. (1991) also reported the oxidation of methionine to sulfoxide and the replacement of the peptide bond with its ketomethylene isoester (COCH₂) to facilitate transport of peptides through the cell membrane. Hildt and Schmidt (EP1127133) describe a peptide derived from Hepatitis Virus B protein which is capable of mediating cell permeability of a protein, peptide, or DNA it is mixed with or coupled to. These are just some of the known modifications and derivatives that are well within the skill of those in the art.

The methods of mediating cell permeability for a protein, peptide or nucleic acid molecule described hereinabove and hereinbelow may of course be used in the preparation of the antisense oligonucleotides and interfering RNA molecules of the invention that are described hereinabove. The ability of such molecules to cross membranes will of course enhance their ability to reach their target which is typically located inside of a cell.

U.S. Pat. No. 5,149,782 discloses conjugating a molecule to be transported across the cell membrane with a membrane blending agent such as fusogenic polypeptides, ion-channel forming polypeptides, other membrane polypeptides, and long chain fatty acids, e.g. myristic acid, palmitic acid. These membrane blending agents insert the molecular conjugates into the lipid bilayer of cellular membranes and facilitate their entry into the cytoplasm.

Low et al., U.S. Pat. No. 5,108,921, reviews available methods for transmembrane delivery of molecules such as, but not limited to, proteins and nucleic acids by the mechanism of receptor mediated endocytotic activity. These receptor systems include those recognizing galactose, mannose, mannose 6-phosphate, transferrin, asialoglycoprotein, transcobalamin (vitamin B₁₂), α-2 macroglobulins, insulin and other peptide growth factors such as epidermal growth factor (EGF). Low et al. teaches that nutrient receptors, such as receptors for biotin and folate, can be advantageously used to enhance transport across the cell membrane due to the location and multiplicity of biotin and folate receptors on the membrane surfaces of most cells and the associated receptor mediated transmembrane transport processes. Thus, a complex formed between a compound to be delivered into the cytoplasm and a ligand, such as biotin or folate, is contacted with a cell membrane bearing biotin or folate receptors to initiate the receptor mediated trans-membrane transport mechanism and thereby permit entry of the desired compound into the cell.

Methods for introducing proteins into cells are described, e.g., in EP1127133 and WO0026379, which publications are included herein in their entirety by reference.

As will be appreciated by those of skill in the art of peptides, the peptide inhibitors of the CD137 interaction according to the present invention is meant to include peptidomimetic drugs or inhibitors, which can also be rapidly screened for binding to CD137 to design perhaps more stable inhibitors.

The methods of mediating cell permeability for a protein, peptide or nucleic acid molecule described hereinabove may of course be used in the preparation of the antisense oligonucleotides and interfering RNA molecules of the invention that are described hereinabove. The ability of such molecules to cross membranes will of course enhance their ability to reach their target which is typically located inside of a cell.

It will also be appreciated that the same means for facilitating or enhancing the transport of peptide inhibitors across cell membranes as discussed above are also applicable to the CD137 protein or its isoforms themselves as well as other peptides and proteins which exert their effects intracellularly.

As regards the antibodies mentioned herein throughout, the term “antibody” is meant to include polyclonal antibodies, monoclonal antibodies (mAbs), chimeric antibodies, anti-idiotypic (anti-Id) antibodies to antibodies that can be labeled in soluble or bound form, as well as fragments thereof provided by any known technique, such as, but not limited to enzymatic cleavage, peptide synthesis or recombinant techniques, and especially single-chain (sc) antibodies, which have the advantage that they are coded for by a single chain of nucleic acids and may therefore easily be introduced into and expressed in cells.

Polyclonal antibodies are heterogeneous populations of antibody molecules derived from the sera of animals immunized with an antigen. A monoclonal antibody contains a substantially homogeneous population of antibodies specific to antigens, which populations contains substantially similar epitope binding sites. MAbs may be obtained by methods known to those skilled in the art. See, for example Kohler and Milstein, Nature, 256:495-497 (1975); U.S. Pat. No. 4,376,110; Ausubel et al., eds., Harlow and Lane ANTIBODIES: A LABORATORY MANUAL, Cold Spring Harbor Laboratory (1988); and Colligan et al., eds., Current Protocols in Immunology, Greene Publishing Assoc. and Wiley Interscience N.Y., (1992-1996), the contents of which references are incorporated entirely herein by reference. Such antibodies may be of any immunoglobulin class including IgG, IgM, IgE, IgA, IgD, and any subclass thereof. A hybridoma producing a mAb of the present invention may be cultivated in vitro, in situ or in vivo. Production of high titers of mAbs in vivo or in situ makes this the presently preferred method of production.

Chimeric antibodies are molecules of which different portions are derived from different animal species, such as those having the variable region derived from a murine mAb and a human immunoglobulin constant region. Chimeric antibodies are primarily used to reduce immunogenicity in application and to increase yields in production, for example, where murine mAbs have higher yields from hybridomas but higher immunogenicity in humans, such that human/murine chimeric mAbs are used. Chimeric antibodies and methods for their production are known in the art (Cabilly et al., Proc. Natl. Acad. Sci. USA 81:3273-3277 (1984); Morrison et al., Proc. Natl. Acad. Sci. USA 81:6851-6855 (1984); Boulianne et al., Nature 312:643-646 (1984); Cabilly et al., European Patent Application 125023 (published Nov. 14, 1984); Neuberger et al., Nature 314:268-270 (1985); Taniguchi et al., European Patent Application 171496 (published Feb. 19, 1985); Morrison et al., European Patent Application 173494 (published Mar. 5, 1986); Neuberger et al., PCT Application WO 8601533, (published Mar. 13, 1986); Kudo et al., European Patent Application 184187 (published Jun. 11, 1986); Sahagan et al., J. Immunol. 137:1066-1074 (1986); Robinson et al., International Patent Application No. WO8702671 (published May 7, 1987); Liu et al., Proc. Natl. Acad. Sci USA 84:3439-3443 (1987); Sun et al., Proc. Natl. Acad. Sci USA 84:214-218 (1987); Better et al., Science 240:1041-1043 (1988); and Harlow and Lane, ANTIBODIES: A LABORATORY MANUAL, supra. These references are entirely incorporated herein by reference.

An anti-idiotypic (anti-Id) antibody is an antibody, which recognizes unique determinants generally associated with the antigen-binding site of an antibody. An Id antibody can be prepared by immunizing an animal of the same species and genetic type (e.g. mouse strain) as the source of the mAb to which an anti-Id is being prepared. The immunized animal will recognize and respond to the idiotypic determinants of the immunizing antibody by producing an antibody to these idiotypic determinants (the anti-Id antibody). See, for example, U.S. Pat. No. 4,699,880, which is herein entirely incorporated by reference.

Such anti idiotypic antibodies may be used to stimulate growth, proliferation, differentiation and/or activation of hematopoietic stem cells, just like CD137 as described further hereinabove.

The anti-Id antibody may also be used as an “immunogen” to induce an immune response in yet another animal, producing a so-called anti-anti-Id antibody. The anti-anti-Id may be epitopically identical to the original mAb which induced the anti-Id. Thus, by using antibodies to the idiotypic determinants of a mAb, it is possible to identify other clones expressing antibodies of identical specificity.

Accordingly, mAbs generated against the CD137 proteins, analogs, fragments or derivatives thereof, of the present invention may be used to induce anti-Id antibodies in suitable animals, such as BALB/c mice. Spleen cells from such immunized mice are used to produce anti-Id hybridomas secreting anti-Id mAbs. Further, the anti-Id mAbs can be coupled to a carrier such as keyhole limpet hemocyanin (KLH) and used to immunize additional BALB/c mice. Sera from these mice will contain anti-anti-Id antibodies that have the binding properties of the original mAb specific for an epitope of the above CD137 protein, or analogs, fragments and derivatives thereof.

The anti-Id mAbs thus have their own idiotypic epitopes, or “idiotopes” structurally similar to the epitope being evaluated, such as CD137 Ligand extracellular epitopes.

The term “antibody” is also meant to include both intact molecules as well as fragments thereof, such as, for example, Fab and F(ab′)2, which are capable of binding antigen. Fab and F(ab′)2 fragments lack the Fc fragment of intact antibody, clear more rapidly from the circulation, and may have less non-specific tissue binding than an intact antibody (Wahl et al., J. Nucl. Med. 24:316-325 (1983)).

It will be appreciated that Fab and F(ab′)2 and other fragments of the antibodies useful in the present invention may be used for the detection and quantitation of the CD137 protein according to the methods disclosed herein for intact antibody molecules. Such fragments are typically produced by proteolytic cleavage, using enzymes such as papain (to produce Fab fragments) or pepsin (to produce F(ab′)2 fragments).

As mentioned hereinabove, Fab or F(ab′)2 fragments, or antibodies or single chain antibodies or anticalins or Trinectins or the like molecules, that specifically bind to CD137 ligand(s) as expressed upon the cell surface of, e.g., activated T or B cells, or on the surface of stem cells. Fragments may of course also be used as functional CD137 analogs, so that when these molecules are multimerized, they may bind and cross-link CD137 ligands on target cells and thereby stimulate proliferation, growth, differentiation and/or activation of stem cells.

An antibody is said to be “capable of binding” a molecule if it is capable of specifically reacting with the molecule to thereby bind the molecule to the antibody. The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three dimensional structural characteristics as well as specific charge characteristics.

An “antigen” is a molecule or a portion of a molecule capable of being bound by an antibody which is additionally capable of inducing an animal to produce antibody capable of binding to an epitope of that antigen. An antigen may have one or more than one epitope. The specific reaction referred to above is meant to indicate that the antigen will react, in a highly selective manner, with its corresponding antibody and not with the multitude of other antibodies which may be evoked by other antigens.

The antibody of the invention may not only bind the CD137 protein, but also inhibit its biological activity. This may be tested easily by methods known to the person of skill in the art. For instance, adding various amounts of the antibody to cells bearing CD137 or to a solution containing crosslinked CD137 protein will result in a lower stimulating activity observed if that antibody is capable of inhibiting the biological activity of CD137, as compared to irrelevant control antibody. Thus, antibodies can be identified that specifically inhibit CD137 activity. Such antibodies are useful in control assays for CD137 activity. Such antibodies are also useful in inhibiting endogenous or exogenous CD137 activity in mammals, tissues, or cells, preferably stem cells, more preferably hematopoietic stem cells, including peripheral and bone marrow stem cells, most preferably on bone marrow stem cells.

The CD137 proteins of the invention may be produced by any standard recombinant DNA procedure (see for example, Sambrook, et al., 1989 and Ausubel et al., 1987-1995, supra) in which suitable eukaryotic or prokaryotic host cells well known in the art are transformed by appropriate eukaryotic or prokaryotic vectors containing the sequences encoding for the proteins. Accordingly, the present invention also concerns such expression vectors and transformed hosts for the production of the proteins of the invention. As mentioned above, these proteins also include their biologically active analogs, fragments and derivatives, and thus the vectors encoding them also include vectors encoding analogs and fragments of these proteins, and the transformed hosts include those producing such analogs and fragments. The derivatives of these proteins, produced by the transformed hosts, are the derivatives produced by standard modification of the proteins or their analogs or fragments.

The invention is now more fully described and illustrated by the examples as set out below. It is understood that the examples are not limiting the scope of the invention, and that e.g. other receptors or molecules or gene sequences may be cloned according to the teaching of the invention.

### BRIEF DESCRIPTION OF THE FIGURES

FIG. 1 shows the effect of human CD137 on proliferation of peripheral stem cells,

FIG. 2a shows IL-6 secretion induced in murine monocytes by human CD137,

FIG. 2b shows the effects of human CD137 on murine stem cells,

FIG. 3 shows a comparison of CD137- and G-CSF-induced stem cell proliferation,

FIG. 4 shows CD137 effects on the different hematopoietic lineages. RFUs: relative fluorescent units.

FIG. 5 shows CD137 effects on the different hematopoietic lineages in the absence of other growth factors. RFUs: relative fluorescent units

FIG. 6 shows CD137 effects on the myeloid lineage under different growth factor concentrations. RFUs: relative fluorescent units.

FIG. 7 shows a comparison of CD137-induced proliferation and differentiation of the myeloid lineage. RLUs: Relative fluorescent units, RLUs: Relative luminescent units.

FIG. 8 shows a comparison of proliferation of human hematopoietic stem cells brought into contact with COS cells that were either untreated (left), expressing empty vector (middle), or expressing human CD137 (right bar).

Tables 2-4 show data relating to a reconstitution of the hematopoietic system by CD137 in vivo.

### EXAMPLES

### Example 1

### Effect of Human CD137 on Peripheral Stem Cells

Human hematopoietic stem cells were isolated from the bone marrow of healthy donors via their CD34 expression by positive selection using the “Direct CD34 Progenitor Kit” (Miltenyi, Bergisch Gladbach, Germany). 96 well plates were coated with 1 μg/ml of CD137-Fc protein or an equimolar amount of Fc protein (0.5 μg/ml). 10⁵ cells were plated per well and incubated for 3, 5 or 8 days. During the last 12 h the cells were labelled with 0.5 μCi ³H-thymidine. The rate of proliferation was determined with a szintillation counter (Packard, Meriden, Conn.). Each condition was done in triplicates.

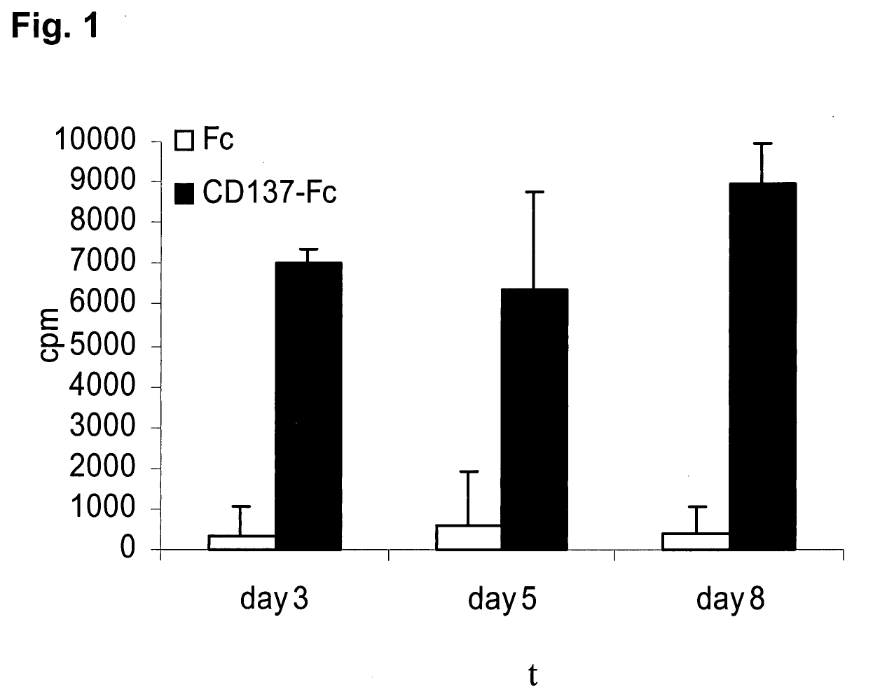

FIG. 1 shows that CD137 is able to induce proliferation of human hematopoietic stem cells. Compared to the Fc control proteins, CD137-Fc protein induces a 8-20 fold increase in DNA synthesis, as evidenced by incorporation of ³H-thymidine. This activity of CD137 is long-lasting, as it is evident at day three, five and eight.

### Example 2

### Effects of Human CD137 on Murine Stem Cells

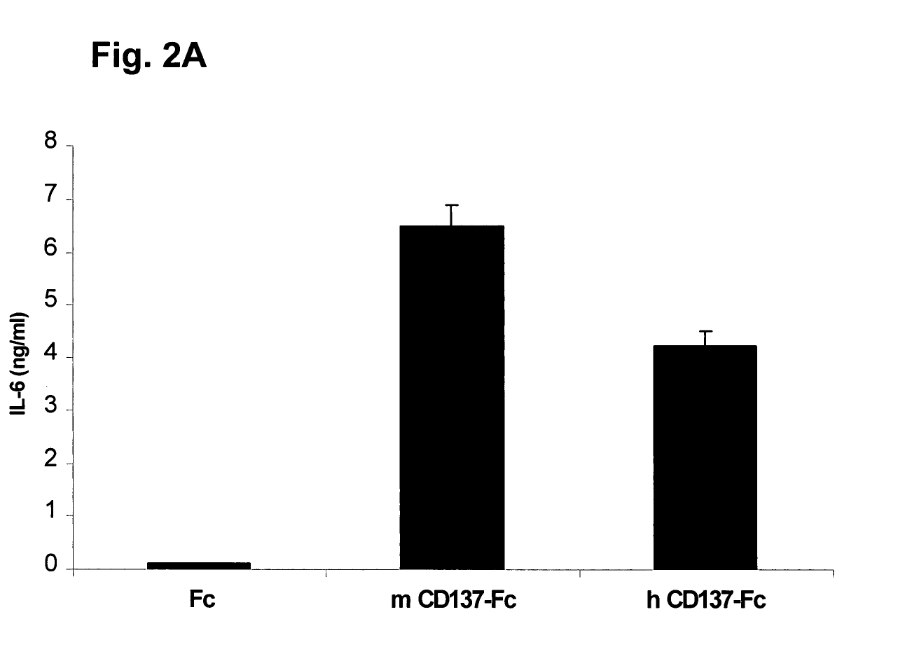

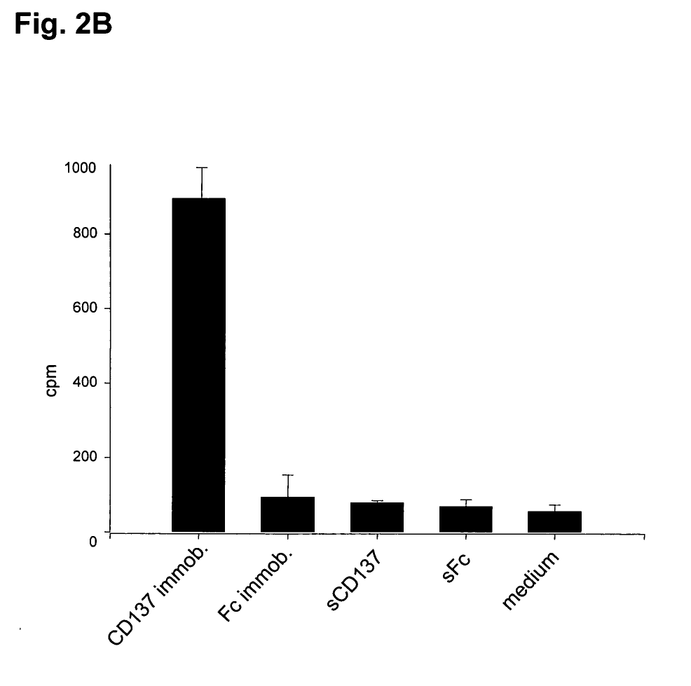

In order to be able to test the effects of human CD137 on murine hematopoietic stem cells, crossreactivity of human CD137 with the murine CD137 Ligand was verified. Human CD137 (h CD137 Fc) induced IL-6 secretion in murine monocytes in the same way as it did in human monocytes (see FIG. 2A for induction by human CD137 Fc on IL-6 secretion of murine monocytes). Also murine CD137-Fc was able to induce IL-6 production in murine monocytes (FIG. 2A, m CD137-Fc). The experiment shown in FIG. 2a was carried out by isolating murine peritoneal exudate cells (>90% monocytes) from the peritoneum of BALB/C mice. 96 well plates were coated with 1 mg/ml of human (h CD137-Fc) or murine CD137-Fc (mCD137-Fc) protein or an equimolar amount of human Fc protein (Fc, 0.5 mg/ml). 10⁵ cells were plated per well and incubated for 16 h. Concentrations of IL-6 in supernatantes were determined by ELISA. Each condition was done in triplicates.

In another experiment, bone marrow cells were isolated from the femur bones of adult NMRI mice. Both ends of the bones were cut and using a syringe the marrow was flushed out with PBS. Tissue particles were removed by a fine-meshed sieve. Flow through cells were washed with PBS. 96 well plates were coated with 1 μg/ml CD137-Fc (CD137 immob) protein or an equimolar amount of Fc (Fc Immob) protein (0.5 μg/ml). Alternatively, both proteins were added as soluble forms (sCD137 and sFc). 5×10⁴ cells were plated per well and cultured for 7 days. Cells were labelled with 0.5 μCi ³H-thymidine during the last 12 h of the experiment. The rate of proliferation was determined with a szintillation counter (Packard, Meriden, Conn.). Each condition was done in triplicates.

FIG. 2b shows that proliferation is induced in murine hematopoietic stem cells (MHSC) isolated from the marrow of the femur bone by human CD137. The rate of proliferation induced is similar to the values seen in FIG. 1. Culture of MHSC on immobilized CD137-Fc protein (CD137 immob.) increased cell proliferation about 10-fold compared to immobilised Fc control protein. In its soluble form, CD137-Fc (sCD137) had no effect on cell proliferation, and neither did soluble Fc control protein (sFc).

### Example 3

### Comparison of CD137 and G-CSF Induced Stem Cell Proliferation

So far Neupogen (G-CSF) is being used to reconstitute the hematopoietic system after chemo- or radiation therapy. Therefore, it was interesting to compare the effects of CD137 and G-CSF on the proliferation of hematopoietic stem cells.

G-CSF is fast acting but its effects last only a few hours (max. 30 h). Due to its short half-life G-CSF is generally administered daily or even several times a day in animal models (Okabe et al., 1990; Aso and Akaza, 1992).

CD137 has a significantly slower kinetics with a slower onset of activity but also a prolonged period of activity. G-CSF was added daily for the first four days whereas CD137 was only given once on the first day of the experiment.

Bone marrow cells were isolated from the femur bones of adult NMRI mice. Both ends of the bones were cut and using a syringe the marrow was flushed out with PBS. Tissue particles were removed by a fine-meshed sieve. Flow through cells were washed with PBS. 96 well plates were coated on day 0 with 50 μg/well of 1.2 μg/ml CD137-Fc protein (=60 ng, CD137-Fc imm.) or an equimolar amount of Fc protein (50 ml of 0.6 μg/ml, Fc imm.). Alternatively, 60 ng of CD137-Fc (Pr. A CD137-Fc) or 30 ng Fc protein (Pr. A Fc) immobilized on protein A by incubation at RT for 1 h were added. Soluble protein A was used, rather that protein A coupled to beads. CD137-Fc and Fc, either immobilized on the plate or immobilized on soluble protein A were added solely on day 1. Human G-CSF (R&D Systems) was added daily from day 1 to 4 at a concentration of 200 ng/ml (G-CSF). 5×10⁴ cells were added per well and cultured for 3, 5 or 7 days. During the last 4 h the cells were labelled with MTS (3-(4,5-dimethydiazol-2-yl)-5-(3-carboxymethoxyphenyl)-2-(4-sufophenyl)-2H-tetrazolium)+PES (phenazine ethosulfat) obtained from Promega. The rate of proliferation was determined at OD₄₉₀. Each condition was done in triplicates.

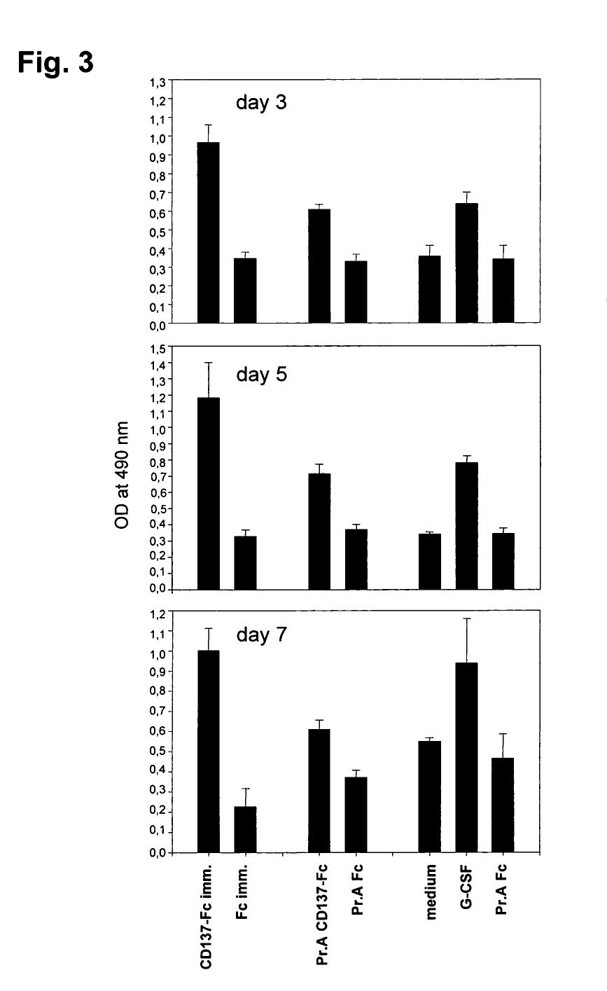

CD137-Fc immobilized on the tissue culture plate had the strongest effect at day 3, 5 and 7 and more than doubled proliferation of the cells (FIG. 3, CD137-Fc imm.). The values for day 7 are an underestimate because the wells with immobilized CD137 were already overgrown by that time and cell growth was subject to contact inhibition.

CD137-Fc, which was immobilized on protein A (FIG. 3, Pr. A CD137-Fc) had about ⅔ of the activity of plate-immobilized CD137-Fc and was as effective as G-CSF (FIG. 3, G-CSF). 1×60 ng CD137-Fc were as or even more active than 4×20 ng G-CSF. The controls, plate-immobilized Fc (Fc imm.) and protein A-immobilized Fc (Pr. A Fc) and soluble Fc were inactive (FIG. 3).

### Example 4

### Evaluation of CD137 Effects on the Different Hematopoietic Lineages

The effects of CD137 on the different hematopoietic lineages were evaluated using a cell based immunoassay, which quantifies expression of lineage-specific markers (CELISA, BioWhittaker, Walkersville, Md., USA).

Progenitors for the myeloid and erythroid lineages were normal human CD34+ cells, which had been isolated from the bone marrow of healthy volunteers by positive selection using the “Direct CD34 Progenitor Kit” (Miltenyi, Bergisch Gladbach, Germany). Progenitors for the megakaryocyte lineage were normal human CD34+ cells, which had been isolated from the peripheral blood of healthy G-CSF-treated volunteers.

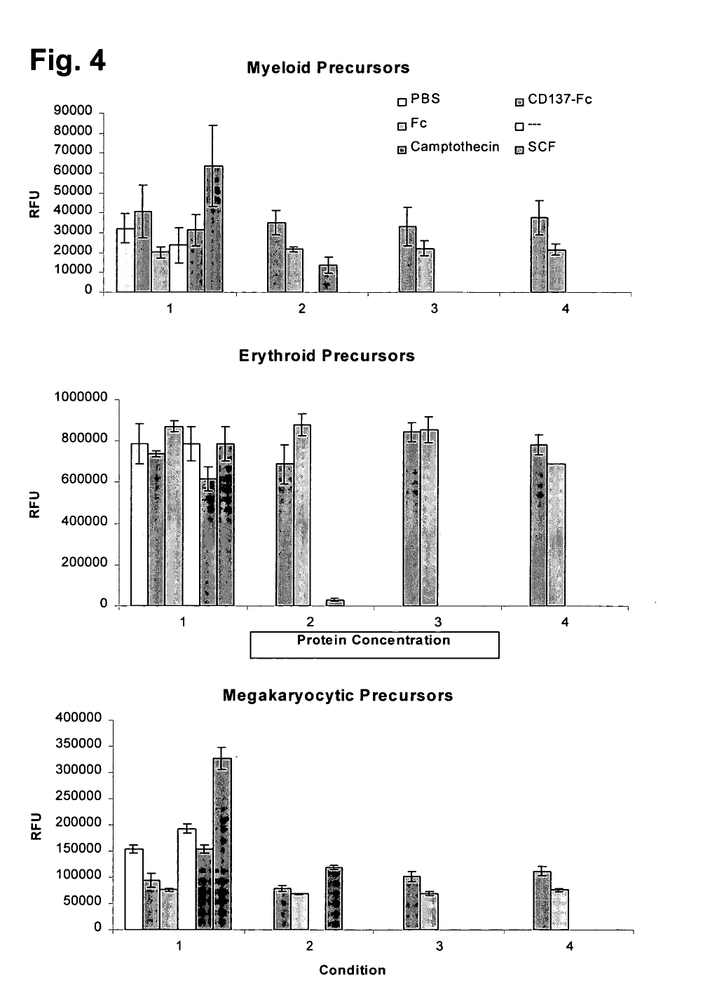

96-well polystyrene plates were precoated with four dilutions of CD137-Fc or Fc control protein at 4° C. overnight or with the dilution buffer PBS. The following additional controls were included in the experiment: no addition; Camptothecin, a cytotoxic compound, at 0.005 μM (condition 1) and at 0.5 μM (condition 2); stem Cell Factor (SCF) at a concentration of 125 ng/ml. The concentrations of the agents in the different conditions shown in FIG. 4 were thus:
Condition 1: (in the Figure, bars indicated with “1”, from left to right) PBS; CD137-Fc 1.2 mg/ml; Fc 0.6 mg/ml; “---”: no addition; Camptothecin 0.005 mM; SCF 125 ng/ml;
Condition 2: (in the Figure, bars indicated with “2”, from left to right) CD137-Fc 0.6 mg/ml; Fc 0.3 mg/ml; Camptothecin 0.5 mM;
Condition 3: (in the Figure, bars indicated with “3”, from left to right) CD137-Fc 0.3 mg/ml; Fc 0.15 mg/ml;
Condition 4: (in the Figure, bars indicated with “4”, from left to right) CD137-Fc 0.15 mg/ml; Fc 0.075 mg/ml.

A volume of 50 μl was used per well. All measurements were done in triplicate. Plates were washed and 2×10³ human CD34+ progenitors were added per well. Cells were cultured in standard cell culture medium (RPMI, 15% fetal calf serum, PenStrep, L-Gln). Lineage specific differentiation to the myeloid, erythroid and megakaryocyte lineage was induced by addition of the factors listed in Table 1.

TABLE 1
Table 1: Lineage specific differentiation was induced in human CD34+
progenitor cells by addition of the listed factors.

| Reagent | Myeloid | Erythroid | Megakaryocyte |
|---|---|---|---|
| SCF | 25 ng/ml | 25 ng/ml | 25 ng/ml |
| GM-CSF | 1 ng/ml | none | 1 ng/ml |
| G-CSF | 1 ng/ml | none | none |
| Transferrin | 200 ug/ml | none | none |
| EPO | None | 3 U/ml | none |
| TPO | None | none | 10 ng/ml |

PBS, no addition (=---) and SCF were only included in condition 1. Camptothecin was only included in condition 1 and 2 (at different concentrations, as described above). Conditions 1, 2, 3 and 4 only refer to different concentrations of CD137 and Fc.

After 10 days of incubation, the cells were transferred to ELISA plates (with 3× multiple washes) for the measurement of expression of lineage-specific cell surface markers. CD11b was used for the myeloid lineage, CD41a (platlet glycoprotein IIb/IIIa) was used for the megakaryocyte lineage and glycophorin A was used for the erythroid lineage.

Results:

The two controls “PBS” and “no-addition” controls gave similar results in all three lineages, indicating that PBS, which was used as a solvent for Fc and CD137-Fc protein had no effect on cell growth.

As expected, the negative control, 0.005 uM Camptothecin showed minimal growth inhibition and at 0.05 uM Camptothecin inhibited growth to a greater extent in all lineages.

The CD137 Fc protein increased proliferation of the myeloid precursor cells at all four concentrations (0.6-0.075 μg/ml).

The CD137-Fc protein had no effect on the growth of erythroid precursors. Both, the CD137-Fc and the Fc control protein reduced growth of the megakaryocytic lineage, implying a toxic effect of the Fc domain. CD137 may have partly counteracted that inhibitory effect.

### Example 5

### Evaluation of CD137 Effects on the Different Hematopoietic Lineages in the Absence of Other Growth Factors

Example 4 had established that CD137 induces proliferation of hematopoietic precursor cells, in particular that of the myeloid and megakaryocyte lineages.

In the experiment depicted in FIG. 4, optimal amounts of growth factors were present (see Table 1 above). These growth factors may be essential cofactors for CD137 activity but their activity may also mask that of CD137. Therefore, growth factors were omitted in Example 5. The cell culture medium contained only 15% fetal calf serum, PenStrep and L-Gln. Otherwise, the experimental set up was identical to that in Example 4.

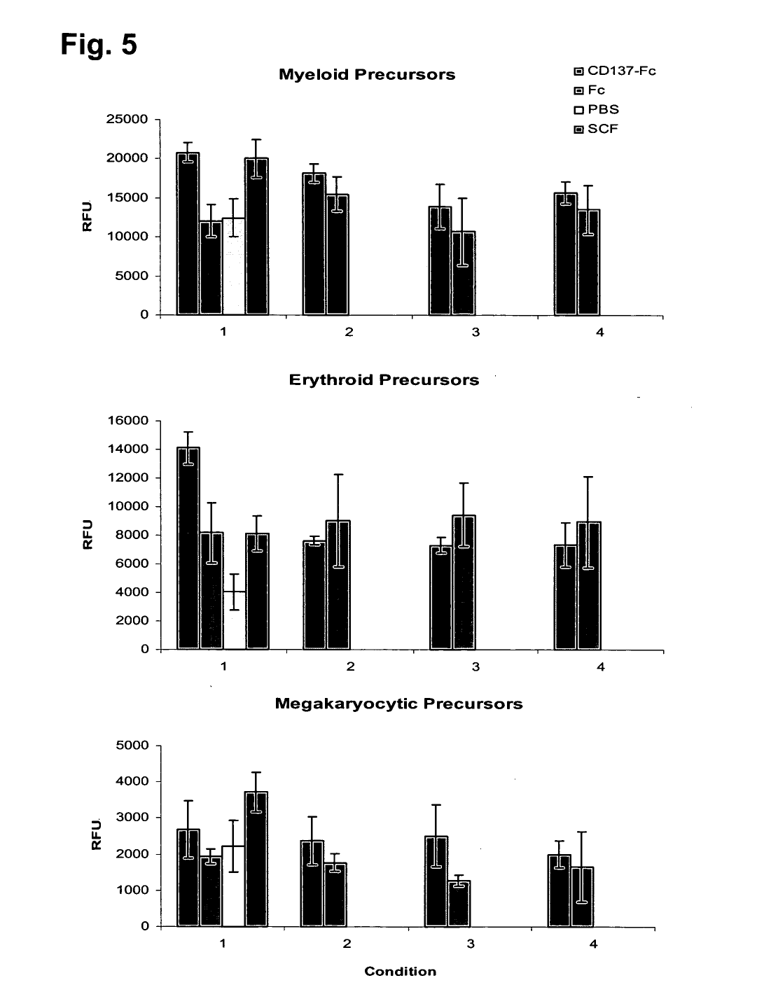

The concentrations of the agents in the different conditions shown in FIG. 5 were thus:
Condition 1: (in the Figure, bars indicated with “1”, from left to right) Fc 0.6 mg/ml; CD137-Fc 1.2 mg/ml; SCF 125 ng/ml; “---”: no addition.
Condition 2: (in the Figure, bars indicated with “2”, from left to right) Fc 0.3 mg/ml; CD137-Fc 0.6 mg/ml;
Condition 3: (in the Figure, bars indicated with “3”, from left to right) Fc 0.15 mg/ml; CD137-Fc 0.3 mg/ml.
Condition 4: (in the Figure, bars indicated with “4”, from left to right) Fc 0.075 mg/ml; CD137-Fc 0.15 mg/ml.

As expected, proliferation of the all three lineages was markedly reduced in the absence of growth factors. CD137 enhanced growth of the myeloid lineage and the megakaryocyte lineage as it has done in the presence of growth factors. In contrast to Example 4, no inhibitory effect of the Fc domain on the megakaryocyte precursors was noticeable. But whereas CD137 had no effect on the growth of the erythroid lineage in the presence of growth factors, it significantly enhanced erythroid proliferation and/or differentiation in the absence of other growth factors. This suggests that the presence of growth factors in Example 4 had masked the activating effect of CD137 on erythroid cells.

These data indicate that CD137 is sufficient for induction of growth and proliferation of the myeloid, megakaryocyte, and erythroid lineage.

### Example 6

### Growth Factor Dependency of CD137 Effects on the Myeloid Lineage

Examples 4 and 5 show that CD137 induces proliferation of myeloid precursor cells, in the presence as well as in the absence of other growth factors. In this example, the influence of growth factor concentrations and potential additive or synergistic effects of these growth factors with CD137 were analysed in more detail. Growth factor concentrations were reduced to 50%, 5% and 0% compared to example 4. Further, only myeloid precursor cells were used. Otherwise the experimental set up was identical to that of the myeloid lineage in example 4.

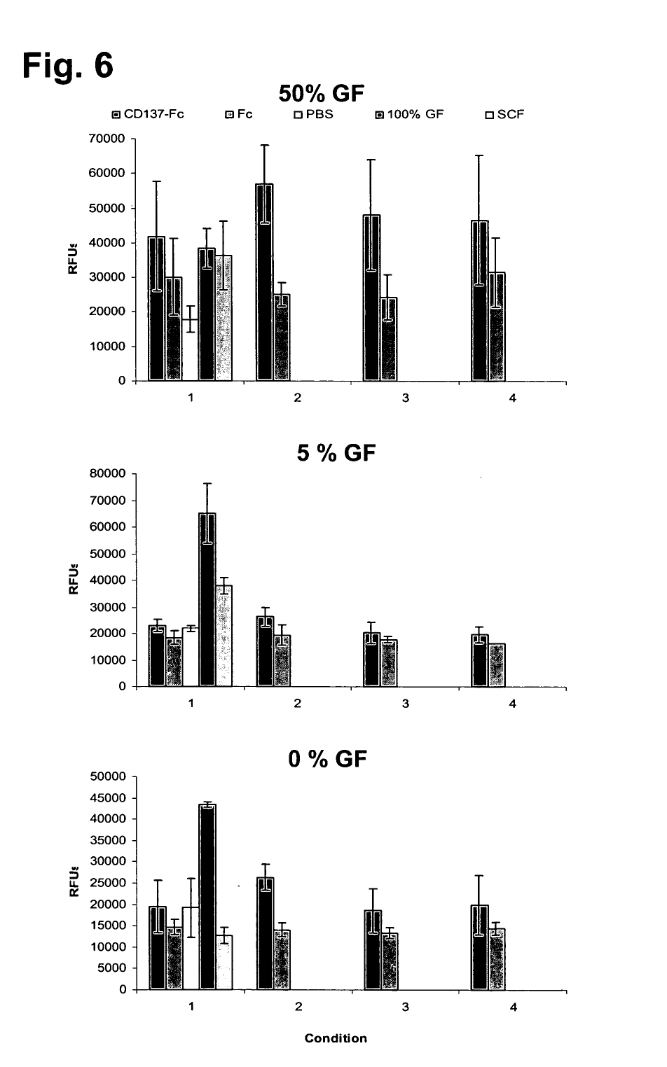

PBS and Fc served as negative controls. The concentrations of the agents in the different conditions shown in FIG. 6 were:
Condition 1: (in the Figure, bars indicated with “1”, from left to right) Fc 0.6 mg/ml; CD137-Fc 1.2 mg/ml. SCF 125 ng/ml.
Condition 2: (in the Figure, bars indicated with “2”, from left to right) Fc 0.3 mg/ml; CD137-Fc 0.6 mg/ml.
Condition 3: (in the Figure, bars indicated with “3”, from left to right) Fc 0.15 mg/ml; CD137-Fc 0.3 mg/ml.
Condition 4: (in the Figure, bars indicated with “4”, from left to right) Fc 0.075 mg/ml; CD137-Fc 0.15 mg/ml.
GF: growth factors as in Table 1.

CD137-Fc enhanced proliferation most significantly when other myeloid growth factors were present (FIG. 6, upper panel). At 50% growth factor concentrations, CD137-Fc was as or even more active as the positive control, stem cell factor (SCF). The same pattern was observed with the other positive control, the 100% growth factor condition (100% GF, 4^{th} bar from the left in condition 1). CD137-Fc not only compensated for the missing 50% growth factors but increased proliferation beyond that obtained with the 100% growth factor control.

Reduction of growth factor concentrations did not result in a stronger induction of growth by CD137. In contrast, the stimulating CD137 effects on myeloid precursors were less prominent at 5% and 0% growth factor concentrations (FIG. 6, middle and lower panel, respectively). These data suggest that although as demonstrated by Example 5 CD137 can induce growth on its own, it does work in combination with one or several of the growth factors listed in Table 1.

### Example 7

### Comparison of CD137-Induced Proliferation and Differentiation

Differentiation was assessed per CELISA (BioWhittaker), measuring the expression of the myeloid-specific protein CD11b. Proliferation was determined using the ViaLight system (BioWhittaker), which measures the amount of ATP in the culture. Cell culture conditions were as described in Example 6. Only myeloid precursors were used.

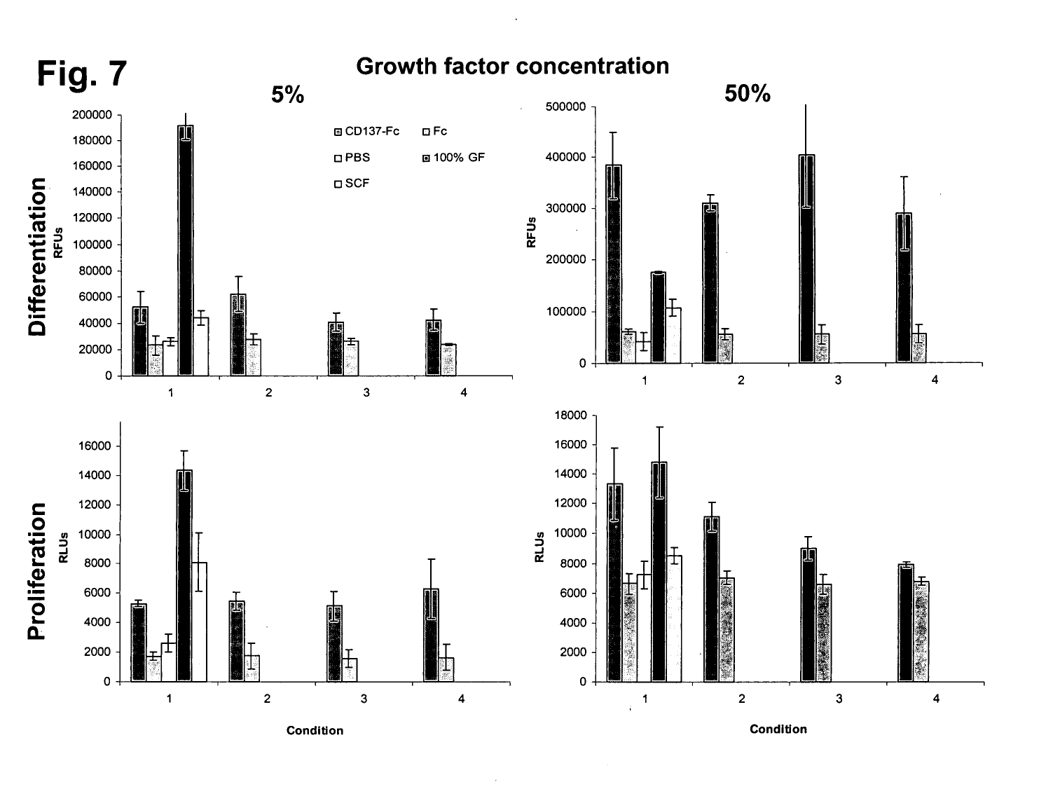

The concentrations of the agents in the different conditions shown in FIG. 7 were:
Condition 1: (in the Figure, bars indicated with “1”, from left to right) Fc 0.6 mg/ml; CD137-Fc 1.2 mg/ml; SCF 125 ng/ml.
Condition 2: (in the Figure, bars indicated with “2”, from left to right) Fc 0.3 mg/ml; CD137-Fc 0.6 mg/ml.
Condition 3: (in the Figure, bars indicated with “3”, from left to right) Fc 0.15 mg/ml; CD137-Fc 0.3 mg/ml.
Condition 4: (in the Figure, bars indicated with “4”, from left to right) Fc 0.075 mg/ml; CD137-Fc 0.15 mg/ml.

At 50% growth factor concentrations (see Table 1 for the list and concentrations of growth factors) the absolute amounts of proliferation and differentiation were larger than at 5%, confirming the results of Example 6, that CD137 works in combination with other growth factors.

At 5% growth factor concentrations the CD137-induced increase in proliferation compared to the Fc control protein was larger than CD137-induced increase in differentiation. Proliferation was increased by CD137 about threefold (from 2.000 to around 6.000 RLUs; FIG. 7, bottom left panel), whereas differentiation was increased about twofold (from 25.000 to around 50.000 RLUs; FIG. 7, top left panel).

The reverse pattern was seen at 50% growth factor concentrations where the relative increase in proliferation was smaller, while the relative increase in differentiation was larger. The comparison is again based on values obtained with the Fc control protein. At 50% growth factor concentrations proliferation was increased by CD137 less than twofold (from 7.000 to 8.000-13.0000 RLUs, depending on the CD137-Fc concentration; FIG. 7, bottom right), whereas differentiation was increased about 7-fold (from 50.000 to around 350.000 RLUs; FIG. 7, top right).

This implies that the rate of differentiation of hematopoietic stem cells increases with increasing growth factor concentrations and thus that differentiation is mainly mediated by the added growth factors. Proliferation on the other hand can be induced by CD137 without much or any contribution from the growth factors. This finding is consistent with data from Example 1-3 where CD137-induced proliferation in the absence of other growth factors.

Nevertheless, CD137 can also contribute significantly to differentiation. The 100% growth factor control induced an about 10-fold higher degree of differentiation compared to 5% growth factor concentrations (200.000 RFUs vs 20.000 in the PBS control; FIG. 7, top left panel). The combination of CD137-Fc and 5% growth factors also resulted in a lower degree of differentiation (50.0000 RFU; FIG. 7, top left panel). However, at 50% of growth factor concentrations CD137 not only compensated for the remaining 50% growth factors but increased differentiation markedly is beyond that level (350.000 vs 200.000 RFUs; FIG. 7, top right panel)

Though CD137-induced proliferation of myeloid precursor cells is not dependent on other growth factors they clearly support it. This is evidenced by the higher proliferation rates at 50% compared to 5% growth factor concentrations (8.000-13.0000 vs 5.000 RLUs, FIG. 7, bottom right vs bottom left panel).

### Example 8

### Effect of CD137-Transfected Cells on Hematopoietic Stem Cells

Human hematopoietic stem cells were isolated from the bone marrow of healthy donors via their CD34 expression by positive selection using the “Direct CD34 Progenitor Kit” (Miltenyi, Bergisch Gladbach, Germany).

COS cells were transfected with a CD137 expression vector (pCD137, containing the entire human CD137 cDNA coding sequence) or the empty vector (pcDNA3), respectively. Untransfected COS cells were used as an additional control. 3×10³ COS cells were seeded into wells of a 96 well plate and grown for 2 days to confluency. Cells were fixed with 0.25% glutaraldehyde for 10 min at room temperature and were then washed twice with PBS. 5×10⁴ human hematopoietic stem cells (HSC) were plated per well and cultured for 7 days. HSC were isolated from the peripheral blood of healthy volunteers via CD34 expression. Cells were labelled with 0.5 μCi ³H-thymidine during the last 12 h of the experiment. The rate of proliferation was determined with a szintillation counter (Packard, Meriden, Conn.). Each condition was done in triplicates.

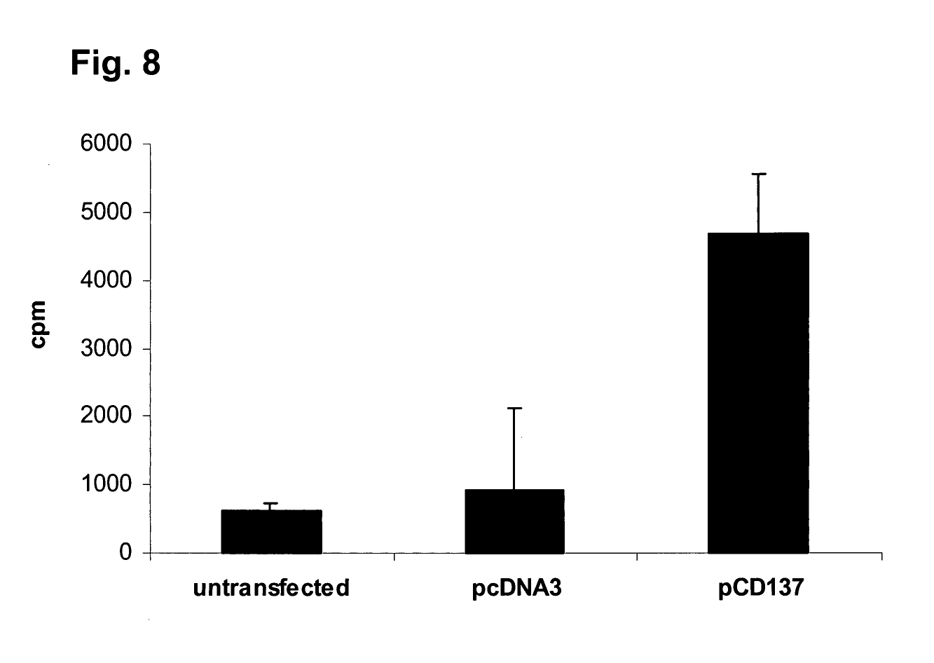

FIG. 8 shows that CD137 when expressed ectopically on transfected cells is able to induce proliferation of human hematopoietic stem cells. Compared to untransfected (untransfected) and vector-transfected (pcDNA3) COS cells, CD137-transfected cells (pCD137) induce a more than 5-fold increase in DNA synthesis, as evidenced by incorporation of ³H-thymidine. This activity of CD137 is long-lasting, as it is evident at day seven.

### Example 9

### In vivo Efficacy of CD137 in Reconstituting the Hematopoietic System

CD137 needs to be immobilized onto a carrier or crosslinked to be functional according to the invention. Protein A was chosen as a carrier for in vivo studies, as it has been effective in crosslinking CD137-Fc in the experiment described in Example 3. CD137-Fc or Fc control protein were immobilized on protein A and injected into NMRI mice. Besides immobilizing CD137-Fe or Fc, protein A has the additional effect of being toxic for cells of the hematopoietic system. This latter activity of protein A exerts a damaging effect on the hematopoietic system of mice as does chemotherapy.

Three mice per group were used. Each mouse received 100 μg of immobilized CD137-Fc or an equimolar amount of immobilized Fc or protein A alone. Mice were killed after 3 weeks and the bone marrow was isolated from the femurs and analysed (Table 2).

TABLE 2
Table 2: Bone marrow composition on day 21.

| | | | | Protein | Protein A |
|---|---|---|---|---|---|
| Cell types | | untreated | Protein A | A-Fc | CD137-Fc |
| R1 | 91% erythroid | 56.15 | 69.18 | 74.51 | 55.67 |
| R2 | 87% lymphoid | 18.21 | 25.35 | 21.95 | 18.54 |
| R3 | 49% undifferentiated blasts | 0.73 | 1.81 | 1.07 | 0.87 |
| | 25% lymphoblasts | | | | |
| | 18% basophil erythroblasts | | | | |
| R4 | 50% undifferentiated blasts | 5.50 | 1.97 | 1.06 | 4.92 |
| | 30% myeloid mainly und. precursors | | | | |
| | 20% erythroid | | | | |
| R5 | 91% granulocytes | 14.21 | 1.12 | 1.33 | 16.17 |
| R6 | 74% monocytes | 5.20 | 0.63 | 0.13 | 3.83 |
| | 20% myeloid precursors | | | | |

Percentage of total cells.
Average, of three mice.

The relative composition of the bone marrow cell population was severely disturbed by the protein A treatment. Crossinking of the Fc control protein onto protein A had no effect on the damaging activity of protein A. The numbers of granulocytes and monocytes were reduced drastically in the protein A (Pr.A) as well as in the Pr.A-Fc mice, demonstrating the myelotoxic effect of protein A. The percentages of these cells in the Pr.A-CD137-Fc mice were far higher (16.17 vs 1.33 and 3.83 vs 0.13%, respectively), and comparable to that of the untreated mice. These data indicate that CD137 induces proliferation of hematopoietic stem cells also in vivo and that it is able to reverse damage to the hematopoietic system. Also, a single application of immobilized CD137 is sufficient for restoration of the hematopoietic system.

In the next experiment NMRI mice were treated with Alkeran, a cytotoxic drug regularly used for human cancer therapy. Alkeran was given i.p. on days 1, 3 and 5. Mice received 100 μg of CD137-Fc immobilized on protein A or equivalent amounts of immobilized Fc control protein. Three mice were used per group. The relative composition of the bone marrow cells (Table 3) and the absolute number of cells in the peripheral blood (Table 4) were determined after 21 days.

TABLE 3
Table 3: Bone marrow composition on day 21.

| | Un- | Alkeran | | Alk. + Pr. | Alk + Pr. A- |
|---|---|---|---|---|---|
| Cell types | treated | (Alk.) | Alk. + Pr. A | A-Fc | CD137-Fc |
| R1 | 51.24 | 60.21 | 77.36 | 71.18 | 52.20 |
| R2 | 18.06 | 22.54 | 19.12 | 15.93 | 19.88 |
| R3 | 0.66 | 0.66 | 0.69 | 0.63 | 0.64 |
| R4 | 5.87 | 2.28 | 1.85 | 3.19 | 3.94 |
| R5 | 19.53 | 11.56 | 0.51 | 8.05 | 18.04 |
| R6 | 4.87 | 2.24 | 0.80 | 1.29 | 5.59 |

Percentage of total cells.
Average of three mice.
Please see Table 2 for key to cell types (R1-R6).

TABLE 4
Table 4: Total number of leukocytes, erythrocytes and thrombocytes in
the peripheral blood per μl.

| | Un- | Alkeran | | Alk. + Pr. | Alk + Pr. A- |
|---|---|---|---|---|---|
| Cell types | treated | (Alk.) | Alk. + Pr. A | A-Fc | CD137-Fc |
| Leukoc. | 1.600 | 830 | 880 | 680 | 1.410 |
| Erythroc | 3.910 | 3.820 | 3.680 | 3.870 | 3.880 |
| Thrombo | 652.000 | 581.000 | 573.000 | 596.000 | 583.000 |

Average of three mice.

Alkeran significantly reduced the percentage of granulocytes (R5) and monocytes (R6) and the toxic effect of protein A-Fc reduced them even further. Again, a single dose of CD137 restored the hematopoietic system. Its therapeutic effect even outlasted two additional doses of Alkeran, given 2 and 4 days after CD137.

In this experiment not only relative but also absolute cell numbers were determined. This excludes the possibility that the increase in the percentage of monocytes or granulocytes may be due to a decrease in other cell types. These in vivo results confirm data in Examples 1 to 8, which show induction of hematopoietic stem cell proliferation by CD137 in vitro.

### REFERENCES

Aso Y, Akaza H. Effect of recombinant human granulocyte colony-stimulating factor in patients receiving chemotherapy for urogenital cancer. Urological rhG-CSF Study Group. J Urol 1992 April; 147(4):1060-4.
Alderson, M. R., CA. Smith, T. W. Tough, T. Davis-Smith, R. J. Armitage, B. Falk, E. Roux, E. Baker, G. R. Sutherland, W. S. Din, and R. G. Goodwin. 1994. Molecular and biological characterization of human 4-1BB and its ligand. Eur. J. Immunol. 24:2219-2227.
DeBenedette, M. A., N. R. Chu, K. E. Pollok, J. Hurtado, W. F. Wade, B. S. Kwon, and T. H. Watts. (1995). Role of 4-1BB ligand in costimulation of T lymphocyte growth and its upregulation on M12 B lymphomas by cAMP. J. Exp. Med. 181:985.
Elias A. D. Dose-intensive therapy for small cell lung cancer. (1995). Chest, 107(6 Suppl): 261S-266S.
Fan D., O'Brian C. A., Ioannides C. G. und Clyne R. K. (1991). Granulocyte-macrophage colony-stimulating factor (GM-CSF) in the management of cancer. In Vivo, 5(6):571-7.
Goodwin, R. G., W. S. Din, T. Davis-Smith, D. M. Anderson, S. D. Gimpel, T. A. Sato, C. R. Maliszewski, C. I. Brannan, N. G. Copeland, N. A. Jenkins, et-al. 1993. Molecular cloning of a ligand for the inducible T cell gene 4-1BB: a member of an emerging family of cytokines with homology to tumor necrosis factor. Eur. J. Immunol. 10:2631-2641.
Hofstra L. S., de Vries E. G., Uyl de Groot C. A. und Vellenga E. (1996). Clinical role of GM-CSF in neutrophil recovery in relation to health care parameters. Med. Oncol., 13(3):177-84.
Jiang Y, Jahagirdar B N, Reinhardt R L, Schwartz R E, Keene C D, Ortiz-Gonzalez X R, Reyes M, Lenvik T, Lund T, Blackstad M, Du J, Aldrich S, Lisberg A, Low W C, Largaespada D A, Verfaillie C M. (2002). Pluripotency of mesenchymal stem cells derived from adult marrow. Nature 418:41-49.
Kwon, B. S. and S. M. Weissman. (1989). cDNA sequences of two inducible T-cell genes. Proc Natl Acad Sci U.S.A. 86:1963-1967.
Langstein J., J. Michel, J. Fritsche, M. Kreutz, R. Andreesen, and H.

Schwarz. 1998. CD137, (ILA/4-1BB), a member of the TNF receptor family regulates monocyte activation via reverse signaling. J. Immunol. 160:2488-94.
Langstein, J., and H. Schwarz. 1999a. Identification of CD137 as a potent monocyte survival factor. J. Leuk. Biol. 65:829.
Langstein, J., J. Michel, and H. Schwarz. 1999b. CD137 Induces Proliferation and Endomitosis in Monocytes. Blood 94:3161.
Lee Y, Gotoh A, Kwon H J, You M, Kohli L, Mantel C, Cooper S, Hangoc G, Miyazawa K, Ohyashiki K, Broxmeyer H E. Enhancement of intracellular signaling associated with hematopoietic progenitor cell survival in response to SDF-1/CXCL12 in synergy with other cytokines. Blood. 2002 Jun. 15;99(12):4307-17.
Michel J., J. Langstein, F. Hofstädter, and H. Schwarz. 1998. A soluble form of CD137 (ILA/4-1BB) is released by activated lymphocytes and is detectable in sera of patients with rheumatoid arthritis. Eur. J. Immunol. 28:290-295.
Neidhart J. A., Hematopoietic colony-stimulating factors. (1992). Uses in combination with standard chemotherapeutic regimens and in support of dose intensification. Cancer, 70:913-20.
Okabe M, Asano M, Kuga T, Komatsu Y, Yamasaki M, Yokoo Y, Itoh S, Morimoto M, Oka T. In vitro and in vivo hematopoietic effect of mutant human granulocyte colony-stimulating factor. Blood. 1990 May 1;75(9): 1788-93.
Pollok, K. E., Y. J. Kim, Z. Zhou, J. Hurtado, K. K. Kim, R. T. Pickard, and B. S. Kwon. 1993. Inducible T cell antigen 4-1BB. Analysis of expression and function. J. Immunol. 150:771-781.
Rozenfeld-Granot G, Toren A, Amariglio N, Nagler A, Rosenthal E, Biniaminov M, Brok-Simoni F, Rechavi G.MAP kinase activation by mu opioid receptor in cord blood CD34(+)CD38(−) cells. Exp Hematol. 2002 May; 30(5):473-80.
Sachs L. (1996). The control of hematopoiesis and leukemia: from basic biology to the clinic. Proc. Natl. Acad. Sci. USA, 93(10):4742-9.
Sambrook, J., Fritsch, E F, and Maniatis, T. (1989) Molecular Cloning, A Laboratory Manual, 2nd ed., Cold Spring Harbor (NY) Laboratory Press
Schwarz, H., J. Tuckwell, and M. Lotz. 1993. A receptor induced by lymphocyte activation (ILA): a new member of the human nerve growth factor/tumor necrosis factor receptor family. Gene. 134:295-298.
Schwarz, H., J. Valbracht, J. Tuckwell, J. Kempis, and M. Lotz. 1995. ILA, the human 4-1BB homologue is inducible in lymphoid and other cell lines. Blood. 85:1043-1052.
Schwarz, H., F. Blanco, J. Valbracht, J. Kempis, and M. Lotz. 1996. ILA, a member of the human NGF/TNF receptor family regulates T lymphocyte proliferation and survival. Blood. 87:2839-2845.
Schwarz H., K. Arden, and M. Lotz. 1997. CD137, a Member of the Tumor Necrosis Factor Receptor Family is Located on Chromosome 1p36, in a Cluster of Related Genes, and Colocalizes with Several Malignancies. Biochem. Biophys. Res. Com. 235:699-703.

1. CD137 or a functional analogue thereof, for use in induction of proliferation of hematopoietic stem cells of a mammal.

2. CD137 or a functional analogue thereof according to claim 1, for use in induction of proliferation of monocytic precursor cells.

3. CD137 or a functional analogue thereof according to claim 1, for use in stimulating hematopoiesis.

4. CD137 or a functional analogue thereof according to claim 1, for use in tissue repair, tissue regeneration and wound healing.

5. CD137 or a functional analogue thereof according to claim 1, for use in enhancing innate and/or adaptive immunity for cancer therapy.

6. CD137 or a functional analogue thereof according to claim 1, for use in enhancing innate and/or adaptive immunity for therapy of infectious disease.

7. CD137 or a functional analogue thereof according to claim 1, for use in enhancing innate and/or adaptive immunity for vaccination against infectious disease.

8. A method of treatment of a disorder characterized by insufficient numbers of cells of the hematopoietic system, including but not limited to T cells, B cells, granulocytes, macrophages, mesenchymal cells, osteoclasts and multipotent adult progenitor cells, comprising the step of administration to a mammal in need thereof of an effective dose of CD137 or a functional analogue thereof.

9. A method of treatment for a disorder characterized by an insufficient number of cells of the hematopoietic system, including but not limited to T cells, B cells, granulocytes, macrophages, mesenchymal cells, osteoclasts and multipotent adult progenitor cells comprising the step of administration of CD137 or a functional analogue thereof to an isolated culture of stem cells and the transfer of the treated cells to a mammal in need thereof.

10. A method for the treatment according to claim 8 of a mammal in need thereof wherein the mammal is characterized by having a decrease in the number or activity of its white blood cells.

11. A method according to claim 10, wherein the decrease is caused by or associated with an immunodeficiency.

12. A method according to claim 11, wherein the immunodeficiency is selected from the group comprising AIDS, hyperimmunoglobulin M syndrome, radiation-induced immunodeficiency, chemotherapy-induced immunodeficiency.

13. A method according to claim 10, wherein the decrease is associated with chemo- and/or radiotherapy and/or removal of blood progenitor cells.

14. A method according to claim 13, wherein the chemo- and/or radiotherapy and/or removal of blood progenitor cells is administered to treat cancer.

15. A method according to claim 13, wherein the chemo- and/or radiotherapy and/or removal of blood progenitor cells is administered to treat autoimmune disease.

16. A method of claim 10, wherein the decrease is associated with leukopenia.

17. A method of claim 16, wherein the leukopenia is caused by a condition selected from the group comprising severe trauma, blood loss, immunodeficiency, or disease such as agranulocytosis or bone marrow failure, wherein said Bone marrow failure may for instance be due to congenital factors, toxins such as benzene, street drugs, viral infections such as hepatitis, or side effects of immunotherapies.

18. A method of claim 16, wherein the leukopenia is caused by a disease such as anemia, aplastic anemia, megablastic anemia, myelophthisic anemia, myelodysplastic syndrome or hairy cell leukemia.

19. A method according to claim 8, wherein the dose of CD137 protein administered is within the range of 1 ng/kg to 1 mg/kg, more preferably 50 ng/kg to 500 μg/kg.

20. A method according to claim 19, wherein the dose of CD137 protein administered is within the range 100 ng/kg to 100 μg/kg more preferably 500 ng/kg to 50 μg/kg.

21. A method according to claim 20, wherein the dose of CD137 protein administered is within the range of 1 to 10 μg/kg more preferably 4 to 6 μg/kg.

22. A method for the stimulation of growth, proliferation, differentiation and/or activation of hematopoietic stem cells, comprising the step of contacting the cells with an effective amount of CD137, during a time period sufficient to allow for said the stimulation of growth, proliferation, differentiation and/or activation.

23. A method of claim 22, wherein the concentration of CD137 is from about 1 ng/ml to about 1 mg/ml.

24. A method of claim 23, wherein the concentration of CD137 is from about 5 ng/ml to about 400 mg/ml.

25. A method of claim 24, wherein the concentration of CD137 is from about 15 ng/ml to about 100 ng/ml.

26. A method of claim 25 wherein the concentration of CD137 is about 60 ng/ml.

27. A method of claim 8, wherein the CD137 or functional analogue thereof is CD137, or a part of CD137, fused to Fc.

28. A method of claim 27 wherein the CD137 contains the extracellular part thereof.

29. A method of claim 28 wherein the CD137 contains amino acids 18 to 255 thereof.

30. A method of claim 29 wherein the CD137 contains amino acids 18 to 186 thereof.

31. A method of claim 8 wherein the CD137 or functional analogue thereof is used in combination with a growth factor.

32. The method of claim 31 wherein the growth factor is selected from among G-CSF, M-CSF, GM-CSF, IL-3, IFN-gamma, TNF, LIF, flt-3, c-kit.

33. The method of claim 32 wherein the growth factor is selected from among G-CSF, M-CSF and GM-CSF.

34. The method of claim 33 wherein the growth factor is G-CSF.

35. The method of claim 8 wherein the CD137 or functional analogue thereof is administered as a single dose.

36. A CD137 molecule, or functional analogue thereof, which is multimerized, for use in a method of claim 8.

37. The molecule of claim 36, wherein the multimer comprises 2 to 20 monomers.

38. The molecule of claim 37, wherein the multimer comprises 3 to 10 monomers.

39. The molecule of claim 37, wherein the monomers are expressed a fusion protein.

40. The molecule of claim 37 wherein the monomers are fused together by means of a covalent bond.

41. A molecule capable of crosslinking CD137 ligand(s) expressed on the surface of a target cell, for use according to claims 1.

42. The molecule of claim 41 which is an antibody or derived from an antibody.

43. The molecule of claim 41 which is an anticalin or derived from an anticalin.

44. The molecule of claim 41 which is a Trinectin or derived from a Trinectin.

45. The molecule of claim 36, characterized by the ability to stimulate the growth, proliferation, differentiation and/or activation of stem cells.

46. The molecule of claim 45, wherein the stem cells are hematopoietic stem cells.

47. The molecule of claim 45, wherein the stem cells are CD34 positive cells.

48. A composition comprising CD137 or a functional analogue thereof, and a diluent and/or carrier.

49. The composition of claim 48, for dermal, transdermal, oral, intravenous, intraperitoneal, intramuscular, or intraliquoreal administration.

50. The composition of claim 48, for use in the treatment of a disorder of a mammal wherein the disorder is associated with decreased number or activity of white blood cells.

51. The composition of claim 50, wherein the decrease is caused by or associated with an immunodeficiency.

52. The composition of claim 51, wherein the immunodeficiency is selected from the group comprising AIDS, hyperimmunoglobulin M syndrome, radiation-induced immunodeficiency, chemotherapy-induced immunodeficiency.

53. A composition of claim 48 for use in stimulation of proliferation and/or differentiation of hematopoietic stem cells as may be beneficial in disorders such as immunodeficiency, agranulocytosis, bone marrow failure, anemia, aplastic anemia, megablastic anemia, myelophthisic anemia, myelodysplastic syndrome or hairy cell leukemia.

54. A method of treatment of a mammal wherein a therapeutically effective amount of a composition of claim 48 is administered to a mammal in need thereof, the mammal having a disorder associated with decreased proliferation and/or differentiation of stem cells.

55. The method of claim 54 wherein the disorder is cancer of cells or the hematopoietic lineage.

56. The method or composition of claim 55, wherein the disorder is leukemia.

57. CD137 or a functional analogue thereof, for use in induction of proliferation of stem cells of a mammal.

58. A method for the treatment of a mammal suffering from a disorder or condition selected from the group consisting of immunodeficiency, agranulocytosis, bone marrow failure, anemia, aplastic anemia, megablastic anemia, myelophthisic anemia, myelodysplastic syndrome and hairy cell leukemia, comprising administering an effective amount of a composition in accordance with claim 57.

59. A molecule capable of crosslinking CD137 ligand(s) expressed on the surface of a target cell, for use in a method of claim 8.

60. The composition of claim 48 wherein the disorder is cancer of cells of the hematopoietic lineage.
